(19) United States
(12) Patent Application Publication
(10) Pub. No.: US 2022/0331224 A1
(43) Pub. Date: Oct. 20, 2022

(54) ENZYMATIC TARGETING CARIOGENIC BACTERIAL-FUNGAL BIOFILM INTERACTION

(71) Applicant: The Trustees of the University of Pennsylvania, Philadelphia, PA (US)

(72) Inventors: Geelsu Hwang, Philadelphia, PA (US); Hye-Eun Kim, Philadelphia, PA (US); Atul Dhall, Philadelphia, PA (US)

(73) Assignee: The Trustees of the University of Pennsylvania, Philadelphia, PA (US)

(21) Appl. No.: 17/718,922

(22) Filed: Apr. 12, 2022

Related U.S. Application Data
(60) Provisional application No. 63/174,707, filed on Apr. 14, 2021.

(51) Int. Cl. A61K 8/66; C12N 9/24; A61P 1/02; A61Q 11/00

(52) U.S. Cl. CPC A61K 8/66; C12N 9/2488; A61P 1/02; A61Q 11/00; C12Y 302/01024

(57) ABSTRACT

The disclosed subject matter provides compositions and methods for treating dental caries. The composition can include an effective amount of a mannan degrading enzyme. The effective amount of the mannan degrading enzyme can be present to treat dental caries of a subject.

### CROSS-REFERENCE TO RELATED APPLICATIONS

This application claims priority to U.S. Provisional Application Ser. No. 63/174,707, filed on Apr. 14, 2021, which is incorporated by reference herein in its entirety.

### STATEMENT REGARDING FEDERALLY-SPONSORED RESEARCH

This invention was made with government support under DE027970 awarded by the National Institutes of Health. The government has certain rights in the invention.

### BACKGROUND

Dental caries is a common biofilm-dependent disease that can afflict children and adults worldwide. The annual cost of treatment of dental caries exceeds $81 billion in the US. Early childhood caries (ECC), an aggressive form of tooth decay with rampant caries lesions, can be associated with prolonged consumption of fermentable carbohydrates. The microorganisms identified in ECC can belong to Streptococci spp., Candida spp., Lactobacilli spp., Actinomyces spp., and Veillonella spp. Candida albicans, an opportunistic fungal pathogen, can associate with cariogenic Streptococcus mutans to form biofilms associated with ECC. Symbiotic and synergistic interactions between these two kingdoms can reinforce biofilm pathogenesis and the virulence of ECC.

The treatment regimen for ECC can depend on the progression of the disease, the social, behavioral, and medical history of the child, and the child's age. Certain children at high risk can require the early restorative intervention of enamel and cavitated lesions to minimize caries development. Certain surgical treatments under general anesthesia can be required for severe ECC, while this can be traumatic for children. Given the aggressive damage caused by ECC and its characterization as a polymicrobial disease with cross-kingdom consortia that can develop hard-to-remove and highly acidic biofilms, there is a need to strategically develop a reliable measure to effectively prevent cross-kingdom interactions and subsequent biofilm development.

### SUMMARY

The disclosed subject matter provides compositions and methods for treating dental caries. An example composition can include an effective amount of a mannan degrading enzyme. The effective amount of the mannan degrading enzyme can be present to treat the dental caries of a subject.

In certain embodiments, the mannan degrading enzyme can be selected from α-mannosidase, β-mannosidase, β-mannanase, and a combination thereof. In non-limiting embodiments, the effective amount of the mannan degrading enzyme can be from about 0.05 U to about 20 U.

In certain embodiments, the composition can be formulated as a toothpaste, a gel, a solution, a wipe, or combinations thereof. In non-limiting embodiments, the composition can be configured to disrupt a formation and development of a biofilm involved in dental caries without damaging oral soft tissues.

In certain embodiments, the subject can be younger than 6-years old.

The disclosed subject matter provides methods for treating dental caries of a subject. An example method can include administering an effective amount of a mannan degrading enzyme to a mouth of the subject. The effective amount is present to treat dental caries of the subject. In non-limiting embodiments, the method can further include contacting the effective amount of a mannan degrading enzyme with a target tooth of the subject for about 5 minutes.

In certain embodiments, the mannan degrading enzyme can be selected from the group consisting of α-mannosidase, β-mannosidase, β-mannanase, and a combination thereof. In non-limiting embodiments, the effective amount of the mannan degrading enzyme can be from about 0.05 U to about 20 U.

In certain embodiments, the mannan degrading enzyme can be formulated as a toothpaste, a gel, a solution, a wipe, or combinations thereof.

In certain embodiments, the formation and development of a biofilm involved in dental caries can be disrupted without damaging oral soft tissues. In non-limiting embodiments, the pH of the mouth can be about 6 after administering the mannan degrading enzyme.

In certain embodiments, the mannan degrading enzyme is administered at least twice daily. In non-limiting embodiments, the mannan degrading enzyme is administered daily to the mouth of the subject for about three weeks.

In certain embodiments, the administering the effective amount of the mannan degrading enzyme can treat the dental caries of the subject without proliferative changes, inflammatory responses, and/or necrosis.

The disclosed subject matter will be further described below.

### BRIEF DESCRIPTION OF THE DRAWINGS

For a more complete understanding of the present disclosure and its features and advantages, reference is now made to the following description, taken in conjunction with the accompanying drawings. The patent or application file contains at least one drawing executed in color. Copies of this patent or patent application publication with color drawing(s) will be provided by the Office upon request and payment of the necessary fee.

FIGS. 1A-1C provide graphs showing the effects of mannan degrading enzymes (MDEs) on the cell wall of C. albicans and its binding potential with GtfB in accordance with the disclosed subject matter.

FIGS. 2A-2E provide graphs and images showing the efficacy of MDEs against S. Mutans-C. albicans biofilms in accordance with the disclosed subject matter.

FIGS. 3A-3D provide graphs showing the binding forces of GtfB to MDE-treated C. albicans in accordance with the disclosed subject matter.

FIGS. 4A-4C provide graphs showing the toxicity assay of MDEs on microbes and human gingival keratinocytes in accordance with the disclosed subject matter.

FIGS. 5A-5F provide graphs showing the activity profiles for MDEs in the MES buffer in accordance with the disclosed subject matter.

FIGS. 6A-6C provide graphs and images showing the effect of MDE treatment on the mechanical stability of S. mutans-C. albicans biofilms in accordance with the disclosed subject matter.

FIGS. 7A-7E provide graphs and images showing the demineralization of human enamel surface by S. mutans-C. albicans biofilms with or without β-mannanase treatment in accordance with the disclosed subject matter.

FIGS. 8A-8C provide graphs showing the enzyme activity in IVIES vs. recommended buffer in accordance with the disclosed subject matter.

FIGS. 9A-9F provide graphs showing the activity profiles for MDEs in saliva in accordance with the disclosed subject matter.

FIG. 10 provides a diagram showing the measurements of the antibiofilm activity of MDEs in saliva in accordance with the disclosed subject matter.

FIGS. 11A-11D provide graphs showing the quantification of biovolumes for S. mutans, C. albicans, and EPS with MDE treatment in accordance with the disclosed subject matter.

FIGS. 12A-12C provide graphs showing the efficacy of MDEs against S. mutans-C. albicans biofilms on human enamel slab in accordance with the disclosed subject matter.

FIGS. 13A-13F provide graphs showing the growth kinetics of S. mutans and C. albicans after treatment with MDEs in accordance with the disclosed subject matter.

FIGS. 14A-14C provide graphs showing the toxicity assay of MDEs on microbes at different units in accordance with the disclosed subject matter.

FIGS. 15A-15D provide graphs showing the efficacy of MDEs against S. mutans-C. albicans biofilms formed with reference (UA159) strain or clinical isolates (PDM1 or PDM4) of S. mutans in accordance with the disclosed subject matter.

FIG. 16 provides a graph showing the toxicity assay of 5-fold of the optimal unit of MDEs on human gingival keratinocytes in accordance with the disclosed subject matter.

FIGS. 17A-17D provide graphs showing the growth of S. mutans and S. gordonii and pH changes over time in accordance with the disclosed subject matter.

FIG. 18 provides a diagram showing the experimental design and treatment regimen for testing the efficacy of topical MDEs against a biofilm-associated oral disease (tooth decay) using in vivo model in accordance with the disclosed subject matter.

FIG. 19 provides a graph showing the therapeutic efficacy of topical MDEs against a biofilm-associated oral disease (tooth decay) in vivo (Larson's modification of Keyes's scoring system) in accordance with the disclosed subject matter.

FIG. 20 provides images showing the effects of topical MDEs on oral soft tissue in vivo after 21 days of treatment in accordance with the disclosed subject matter.

It is to be understood that both the foregoing general description and the following detailed description are exemplary and are intended to provide further explanation of the disclosed subject matter.

### DETAILED DESCRIPTION

The disclosed subject matter provides techniques for treating dental caries. The disclosed techniques can be used for disrupting bacterial-fungal interaction associated with dental caries using mannan-degrading enzymes. The disclosed techniques can treat dental caries without affecting the composition of the microbiome and damaging oral soft tissues.

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. All publications, patent applications, patents, and other references mentioned herein are incorporated by reference in their entirety. The materials, methods, and examples disclosed herein are illustrative only and not intended to be limiting.

As used in the specification and the appended claims, the singular forms “a,” “an,” and “the” include plural referents unless the context clearly dictates otherwise. Thus, for example, a reference to “a compound” includes mixtures of compounds.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value.

As used herein, the term “administering” can mean any suitable route, e.g., via topical administration, intraocular administration, or periocular administration without limitation to other routes of administration.

The term “effective amount,” as used herein, refers to the amount of active agent sufficient to treat, prevent, or manage a disease. Further, a therapeutically effective amount with respect to the second targeting probe of the disclosure can mean the amount of active agent alone, or in combination with other therapies, that provides a therapeutic benefit in the treatment or management of the disease, which can include a decrease in the severity of disease symptoms, an increase in frequency and duration of disease symptom-free periods, or a prevention of impairment or disability due to the disease affliction. The term can encompass an amount that improves overall therapy, reduces or avoids unwanted effects, or enhances the therapeutic efficacy of or synergies with another therapeutic agent.

An “individual” or “subject” herein is a vertebrate, such as a human or non-human animal, for example, a mammal. Mammals include, but are not limited to, humans, primates, farm animals, sport animals, rodents, and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

The terms “treat,” “treating” or “treatment,” and other grammatical equivalents as used herein, include alleviating, abating, ameliorating, or preventing a disease, condition or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder.

The disclosed subject matter provides a composition for treating dental caries. In certain embodiments, the composition can include an effective amount of a mannan degrading enzyme for treating dental caries of a subject. In non-limiting embodiments, the mannan degrading enzyme can be α-mannosidase, β-mannosidase, β-mannanase, or combinations thereof.

In certain embodiments, the effective amount of the mannan degrading enzyme is from about 0.05 μmol/min (U) to about 20 U. For example, the composition can include α-mannosidase having an enzyme activity from about 0.05 U to about 1 U, from about 0.1 U to about 1 U, from about 0.15 U to about 1 U, from about 0.2 U to about 1 U, from about 0.25 U to about 1 U, from about 0.5 U to about 1 U, from about 0.75 U to about 1 U, from about 0.05 U to about 0.75 U, from about 0.1 U to about 0.75 U, from about 0.2 U to about 0.75 U, from about 0.25 U to about 0.75 U, from about 0.5 U to about 0.75 U, from about 0.05 U to about 0.5 U, from about 0.1 U to about 0.5 U, from about 0.2 U to about 0.5 U, from about 0.25 U to about 0.5 U, from about 0.05 U to about 0.25 U, from about 0.1 U to about 0.25 U, from about 0.2 U to about 0.25 U, from about 0.05 to about 0.25 U, from about 0.1 to about 0.25 U, from about 0.05 to about 0.2 U, from about 0.1 to about 0.2 U, or from about 0.05 to about 0.1 U.

In non-limiting embodiments, the composition can include β-mannosidase having an enzyme activity from about 0.05 U to about 1 U, from about 0.1 U to about 1 U, from about 0.15 U to about 1 U, from about 0.2 U to about 1 U, from about 0.25 U to about 1 U, from about 0.5 U to about 1 U, from about 0.75 U to about 1 U, from about 0.05 U to about 0.75 U, from about 0.1 U to about 0.75 U, from about 0.2 U to about 0.75 U, from about 0.25 U to about 0.75 U, from about 0.5 U to about 0.75 U, from about 0.05 U to about 0.5 U, from about 0.1 U to about 0.5 U, from about 0.2 U to about 0.5 U, from about 0.25 U to about 0.5 U, from about 0.05 U to about 0.25 U, from about 0.1 U to about 0.25 U, from about 0.2 U to about 0.25 U, from about 0.05 to about 0.25 U, from about 0.1 to about 0.25 U, from about 0.05 to about 0.2 U, from about 0.1 to about 0.2 U, or from about 0.05 to about 0.1 U.

In non-limiting embodiments, the composition can include β-mannanase having an enzyme activity from about 0.5 U to about 20 U, from about 0.75 U to about 20 U, from about 1 U to about 20 U, from about 2 U to about 20 U, from about 5 U to about 20 U, from about 10 U to about 20 U, from about 0.5 U to about 10 U, from about 0.75 U to about 10 U, from about 1 U to about 10 U, from about 2 U to about 10 U, from about 5 U to about 10 U, from about 0.5 U to about 15 U, from about 0.75 U to about 15 U, from about 1 U to about 15 U, from about 2 U to about 15 U, from about 5 U to about 15 U, from about 0.5 U to about 10 U, from about 0.75 U to about 10 U, from about 1 U to about 10 U, from about 2 U to about 10 U, from about 5 U to about 10 U, from about 0.5 U to about 5 U, from about 0.75 U to about 5 U, from about 1 U to about 5 U, from about 2 U to about 5 U, from about 0.5 U to about 2 U, from about 0.75 U to about 2 U, from about 1 U to about 2 U, from about 0.5 U to about 1 U, from about 0.75 U to about 1 U, or from about 0.5 U to about 0.75 U.

In certain embodiments, the disclosed composition can be formulated for administration into the mouth of a subject. For example, the composition can be formulated as a toothpaste, a gel, a solution, a wipe, or combinations thereof.

In certain embodiments, the composition can be configured to disrupt a formation and development of a biofilm involved in dental caries. For example, the composition can be configured to treat dental caries by reducing or disrupting a biofilm created by a fungus and a cariogenic bacterium. Certain interactions between a fungus (e.g., Candida albicans) and a cariogenic bacterium (e.g., Streptococcus mutans) can promote the development of hard-to-remove and highly acidic biofilms exacerbating the virulence. These interactions can be mediated via glucosyltransferases (GtfB) binding-to-mannans on the cell wall of C. albicans. In non-limiting embodiments, the disclosed subject matter can disrupt the mechanical stability of the biofilm using mannan degrading enzymes (e.g., exo- and endo-enzymes). For example, the mannan degrading enzymes can include α-mannosidase, β-mannosidase, β-mannanase, or combinations thereof. The mannan degrading enzymes can decrease in binding forces of GtfB-to-C. albicans (e.g., ˜15-fold reduction) and degrade mannans on C. albicans cell wall. In non-limiting embodiments, the targeted disruption of receptor-ligand at the cellular level can change, affecting biofilm biomass, population, mechanical stability, and/or acidity, culminating with a marked reduction of human tooth-enamel demineralization at the macroscale.

In certain embodiments, the composition can be used to treat dental caries without damaging oral soft tissues. For example, the composition can cause less antimicrobial resistance and toxicity toward adjacent cells in the oral cavity. In non-limiting embodiments, gingival keratinocytes will maintain similar cell viability (e.g., above 90%) after the administration of the disclosed composition.

In certain embodiments, the mannan degrading enzyme can be sustainable in a mouth of a subject. For example, the mannan degrading enzyme can be sustainable in an environment having a pH of about 5, about 6, about 7, or about 8. In non-limiting embodiments, the mannan degrading enzyme can also be sustainable in an environment having a temperature of about 37° C.

In certain embodiments, the disclosed mannan degrading enzymes can be used in combination with other antibacterial or antifungal agents (e.g., fluoride, chlorhexidine, hydrogen peroxide, fluconazole, nystatin). In non-limiting embodiments, the antibacterial or antifungal agents can be used with reduced cytotoxicity and/or concentrations for treating dental caries. The disclosed mannan degrading enzymes, which can disrupt biofilm mechanical stability and reduce human tooth-enamel demineralization without cytotoxic effects, can be used for treating dental caries. The cytotoxicity of the antibacterial/antifungal agents can be reduced by combining the reduced amount of the antibacterial/antifungal agents with the disclosed mannan degrading enzymes. The combined treatment of the mannan degrading enzymes and the antibacterial/antifungal agents can provide combinatorial or synergistic effects on the treatment of dental caries with reduced cytotoxicity.

In certain embodiments, the disclosed subject matter provides a method of treating the dental caries of a subject. The method can include administering an effective amount of a mannan degrading enzyme to a mouth of the subject for treating dental caries of the subject.

In certain embodiments, the subject can be younger than about 15 years old, about 14 years old, about 13 years old, about 12 years old, about 11 years old, about 10 years old, about 9 years old, about 8 years old, about 7 years old, about 6 years old, or about 5 years old.

In certain embodiments, the disclosed composition can be administered to the mouth of the subject to contact the effective amount of a mannan degrading enzyme with a target tooth of the subject. For example, but not by way of limitation, the target tooth and the mannan degrading enzyme can be contacted for at least about 1 minute, about 2 minutes, about 3 minutes, about 4 minutes, about 5 minutes, about 6 minutes, about 7 minutes, about 8 minutes, about 9 minutes, or about 10 minutes. In non-limiting embodiments, the administration can be at least once a day, at least twice a day, at least three times a day, at least four times a day, at least five times a day, at least once a week, at least twice a week, at least once a month, at least twice a month, at least six times a year, at least four times a year, up to twice a day, up to three times a day, up to four times a day, up to five times a day, up to once a week, up to twice a week, up to three times a month, up to six times a year, or up to four times a year. In certain embodiments, the mannan degrading enzyme can be daily administered to the mouth of the subject for about at least one week, about at least two weeks, about at least three weeks, about at least four weeks, about at least five weeks, about at least six weeks, about at least seven weeks, about at least eight weeks, about at least nine weeks, about at least ten weeks, about at least eleven weeks, or about at least twelve weeks. In non-limiting embodiments, the mannan degrading enzyme can be weekly administered to the mouth of the subject for about at least one week, about at least two weeks, about at least three weeks, about at least four weeks, about at least five weeks, about at least six weeks, about at least seven weeks, about at least eight weeks, about at least nine weeks, about at least ten weeks, about at least eleven weeks, or about at least twelve weeks.

In certain embodiments, the mannan degrading enzyme can be α-mannosidase, β-mannosidase, β-mannanase, or combinations thereof. In non-limiting embodiments, the effective amount of the mannan degrading enzyme is from about 0.05 μmol/min (U) to about 20 U. For example, the composition can include α-mannosidase having an enzyme activity from about 0.05 U to about 1 U, from about 0.1 U to about 1 U, from about 0.15 U to about 1 U, from about 0.2 U to about 1 U, from about 0.25 U to about 1 U, from about 0.5 U to about 1 U, from about 0.75 U to about 1 U, from about 0.05 U to about 0.75 U, from about 0.1 U to about 0.75 U, from about 0.2 U to about 0.75 U, from about 0.25 U to about 0.75 U, from about 0.5 U to about 0.75 U, from about 0.05 U to about 0.5 U, from about 0.1 U to about 0.5 U, from about 0.2 U to about 0.5 U, from about 0.25 U to about 0.5 U, from about 0.05 U to about 0.25 U, from about 0.1 U to about 0.25 U, from about 0.2 U to about 0.25 U, from about 0.05 to about 0.25 U, from about 0.1 to about 0.25 U, from about 0.2 to about 0.25 U, from about 0.05 to about 0.2 U, from about 0.1 to about 0.2 U, or from about 0.05 to about 0.1 U. In non-limiting embodiments, the composition can include β-mannosidase having an enzyme activity from about 0.05 U to about 1 U, from about 0.1 U to about 1 U, from about 0.15 U to about 1 U, from about 0.2 U to about 1 U, from about 0.25 U to about 1 U, from about 0.5 U to about 1 U, from about 0.75 U to about 1 U, from about 0.05 U to about 0.75 U, from about 0.1 U to about 0.75 U, from about 0.2 U to about 0.75 U, from about 0.25 U to about 0.75 U, from about 0.5 U to about 0.75 U, from about 0.05 U to about 0.5 U, from about 0.1 U to about 0.5 U, from about 0.2 U to about 0.5 U, from about 0.25 U to about 0.5 U, from about 0.05 U to about 0.25 U, from about 0.1 U to about 0.25 U, from about 0.2 U to about 0.25 U, from about 0.05 to about 0.25 U, from about 0.1 to about 0.25 U, from about 0.2 to about 0.25 U, from about 0.05 to about 0.2 U, from about 0.1 to about 0.2 U, or from about 0.05 to about 0.1 U. In non-limiting embodiments, the composition can include β-mannanase having an enzyme activity from about 0.5 U to about 20 U, from about 0.75 U to about 20 U, from about 1 U to about 20 U, from about 2 U to about 20 U, from about 5 U to about 20 U, from about 10 U to about 20 U, from about 0.5 U to about 10 U, from about 0.75 U to about 10 U, from about 1 U to about 10 U, from about 2 U to about 10 U, from about 5 U to about 10 U, from about 0.5 U to about 15 U, from about 0.75 U to about 15 U, from about 1 U to about 15 U, from about 2 U to about 15 U, from about 5 U to about 15 U, from about 0.5 U to about 10 U, from about 0.75 U to about 10 U, from about 1 U to about 10 U, from about 2 U to about 10 U, from about 5 U to about 10 U, from about 0.5 U to about 5 U, from about 0.75 U to about 5 U, from about 1 U to about 5 U, from about 2 U to about 5 U, from about 0.5 U to about 2 U, from about 0.75 U to about 2 U, from about 1 U to about 2 U, from about 0.5 U to about 1 U, from about 0.75 U to about 1 U, or from about 0.5 U to about 0.75 U.

In certain embodiments, the pH of the mouth can be altered after administering the manna degrading enzyme. For example, the pH of the subject's mouth can be about 5, about 6, or about 7 after administering the mannan degrading enzyme.

In certain embodiments. the administering the effective amount of a mannan degrading enzyme can treat the dental caries of the subject without proliferative changes, inflammatory responses, and/or necrosis.

In certain embodiments, the enzymatic activity disrupted biofilm mechanical stability and significantly can reduce human tooth-enamel demineralization without cytotoxic effects on gingival-keratinocytes. For example, the disclosed exo- and endo-enzymes can reduce the biomass of biofilms without killing microorganisms, alleviating the production of an acidic pH environment conducive to tooth decay. In non-limiting embodiments, the disclosed mannan degrading enzyme can facilitate the removal of biofilm. For example, after or during the administration of the disclosed composition (e.g., mannan degrading enzyme), the biofilm can be removed when it is exposed to fluid shear stress (e.g., mouth wash).

### EXAMPLES

### Example 1: S. mutans-Derived Exoenzyme (GtfB) Effectively Modulates Cross-Kingdom Interactions

Certain secreted S. mutans-derived GtfB can promote the co-existence between S. mutans and C. albicans within biofilms when clinical conditions are conducive to ECC (e.g., sugar-rich exposure). S. mutans-derived GtfB is capable of binding to C. albicans surface and produces large amounts of extracellular glucans on the fungal surface. A confocal image reveals large numbers of S. mutans cells 101 bound to the glucans formed on the yeast cell surface. These EPS formed in situ provide enhanced bacterial binding sites for S. mutans, suggesting that glucans can play a role in mediating their binding and biofilm formation. The GtfB 102 can bind with remarkable strength to the C. albicans surface 103 (˜2 nN) with a highly stable bond. In turn, it plays a key role in modulating the EPS synthesis in situ and the development of virulent co-species biofilms. The presence of C. albicans together with S. mutans can enhance the assembly of an EPS-rich matrix, leading to the development of large biofilms.

As interactions of GtfB with C. albicans appear to be critical for this bacterial-fungal association and enhanced microbial colonization, whether lack of gtfB expression by S. mutans can impair the formation of co-species biofilms in the presence of sucrose was examined. The gtfB mutant co-cultured with C. albicans severely disrupted biofilm development, which was devoid of any microcolonies with minimal EPS-matrix and fewer yeast cells. These observed differences are linked to a defect in insoluble glucan synthesis since supplementation with the GtfB enzyme restores the co-species biofilm phenotype in the presence of igtfB mutants. Altogether, the available in vitro evidence clearly demonstrates that GtfB binding/activity on the C. albicans surface play key roles in modulating the formation of co-species biofilms in an environment conducive to ECC.

C. albicans mannans mediate GtfB binding to modulate cross-kingdom biofilm development: N- and O-linked mannans on the C. albicans cell wall can play key roles in GtfB binding to the fungal surface. Mutant strains defective in mannans showed reduced GtfB binding (vs. wild type), which in turn impaired EPS production and abrogated mixed-species biofilm formation in vivo, revealing potential antibiofilm therapeutic targets. C. Albicans cell wall components can mediate the binding of secreted metabolic byproducts and/or extracellular signaling molecules. Among three major components of the fungal cell wall (mannans, glucans, and chitin), mannans are located at the most outer cell wall layer of C. albicans. S. mutans-derived GtfB binds to yeast, pseudohyphae, and hyphal form of C. albicans, and bound-GtfB retained its enzymatic activity. GtfB can bind in active form to mannans. Thus, these biomolecules can be involved in the binding of GtfB to the fungal cell surface. To determine which of these molecules can be important for GtfB adhesive interactions, well-characterized C. albicans mutants with specific truncations in the wild-type structures of β- and N-linked mannans were selected. The physical binding interactions between the GtfB and C. albicans cell surface via single-molecule force spectroscopy (SMFS-AFM) were evaluated. GtfB can bind strongly to purified mannans, while C. albicans strains defective in β-mannan (pmt4ΔΔ) or N-mannan outer chain (och1ΔΔ) showed severely reduced GtfB binding (vs. wild-type). It indicates that mannans on C. albicans surface can mediate a strong and stable binding of GtfB.

Then, the role of mannosylation in the development of co-species biofilms on the apatitic surface was investigated. GtfB binding to the surface of C. albicans can provide a platform through which GtfB generates EPS that is important for the development of S. mutans-C. albicans cross-kingdom biofilms. Interestingly, mannan-defective mutants were impaired in their ability to form mixed-species biofilms with S. mutans on the apatitic surface. Confocal images of biofilms show clear disruption of C. albicans accumulation and EPS-matrix in the biofilms formed by pmt4ΔΔ or och1ΔΔ (vs. C. albicans WT).

In vivo implications of S. mutans-C. albicans interactions in the pathogenesis of dental caries: whether the presence of C. albicans together with S. mutans enhances the severity of dental caries was evaluated using the rodent model that mimics the clinical conditions found in ECC. In this model, hyposalivatory rats were used while providing a sucrose-rich NIH diet 2000 and sugared water ad libitum. Protracted feeding/drinking and blocking saliva access to teeth support the clinical conditions found in ECC. The animals were infected using the standardized procedures as follows: (1) S. mutans UA159 (Sm)+C. albicans NGY152 (Ca WT), (2) Sm+Ca och1ΔΔ, (3) Sm+revertant of Ca och1ΔΔ, (4) Sm ΔgtfB+Ca WT. The effect of co-infection of S. mutans with C. albicans on both the microbial colonization and the severity of caries in vivo were significant. SEM images revealed that coinfection with C. albicans WT resulted in abundant plaque-biofilm formation over the smooth surface of the teeth. Close-up images show hyphal forms of C. albicans populating the surface of biofilms, similar to the fungal distribution in in vitro biofilm structure. In contrast, C. albicans och1ΔΔ was unable to form cross-kingdom biofilms on the tooth surface of rats co-infected with S. mutans. The level of plaque-biofilm formation was nearly completely recovered when rats were coinfected with S. mutans and the revertant strain of C. albicans och1ΔΔ.

Collectively, the results reveal a key role for C. albicans β-mannan and N-mannan outer chain in (1) mediating GtfB binding, (2) promoting the EPS-matrix assembly, and (3) facilitating mixed-species biofilm development. The in vivo data reveal that the ability of C. albicans deficient in mannan to form mixed-species biofilms with S. mutans can be impaired when compared to wild-type strains. The unveiled mechanism emphasizes the need to target C. albicans mannan by blocking the binding of GtfB to the fungal cell wall.

Abundant GtfB binding/activity on a fungal surface can be important for S. mutans-C. albicans association, providing a platform (e.g., EPS-matrix) for the development of cross-kingdom plaque-biofilms in vivo. Thus, reducing the adhesion of GtfB to the fungal surface by disrupting its binding sites on C. albicans surface can lead to a new paradigm to prevent or treat biofilm-associated hypervirulent oral infectious disease (ECC) without the need of using antimicrobials. Mannan degrading enzymes (MDEs) can be used for specific mannan degradation on Candida cell walls. The disclosed non-microbiocidal and antimicrobial independent approach can target a pathogenic interaction without necessarily perturbing resident microbiota to prevent and control the onset of this costly and difficult-to-treat oral disease.

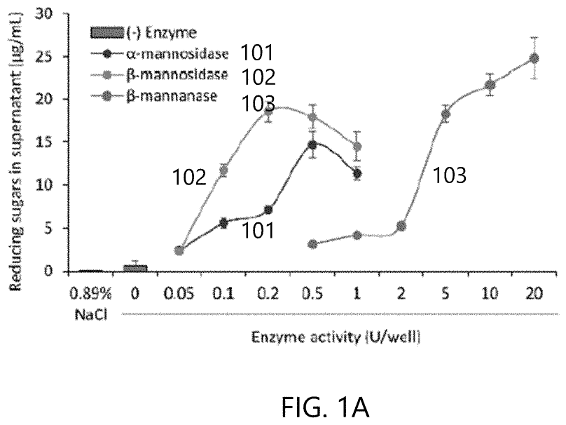

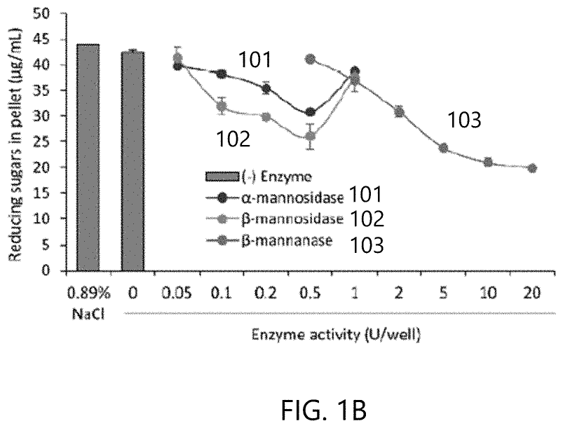

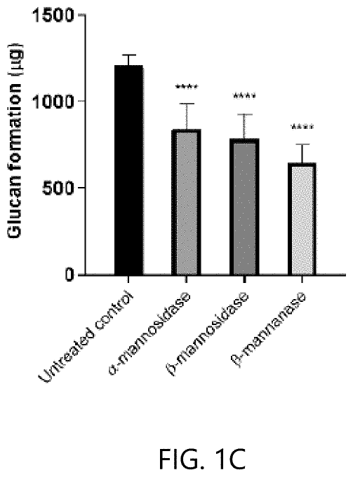

MDEs degrade the C. albicans cell wall and reduce binding potential with GtfB: since the MDEs displayed activity against their respective substrates within 5 min and with the purposes of the disclosed subject matter to develop a feasible therapeutic intervention strategy to limit biofilm interactions in ECC, the optimal treatment time can be about 5 min. After selecting an optimal treatment time, enzymatic cell wall degradation of C. albicans was assessed by calculating the glucose concentration in the supernatant and pellet (μg/mL) after treatment. A dose-dependent increase was detected in supernatant glucose concentration with increasing enzyme units for all MDEs (FIG. 1A). Consequently, there was a similar decrease in glucose concentration in the pellet, indicating reduced mannan components on MDE-treated C. albicans (FIG. 1B). From these results, the optimal enzyme units were determined for cell-wall mannan degradation as 0.5, 0.2, and 10 U/well for α-mannosidase 101, β-mannosidase 102, and β-mannanase 103, respectively.

Mannans on the cell wall of C. albicans can mediate GtfB binding to modulate S. mutans-C. albicans biofilm development. To demonstrate the effect of cell wall degradation on C. albicans to GtfB binding and activity by the use of MDEs, the binding potential of GtfB on the surface of C. albicans was assessed. Each group of C. albicans with or without enzyme treatment was incubated with equal amounts of GtfB and sucrose to compare the amount of glucans formed on C. albicans. C. albicans treated with optimal units of MDEs for 5 min showed decreased glucan formation when compared to the untreated control (FIG. 1C). Overall, β-mannanase was most effective (e.g., ˜50% decrease in glucan formation) followed by 13-mannosidase (e.g., ˜35% decrease) and α-mannosidase (e.g., ˜30% decrease). Results indicate that MDEs degraded the cell wall of C. albicans, and this led to fewer sites available for the binding of GtfB. Subsequently, in the presence of sucrose, this can lead to lower amounts of glucans formed.

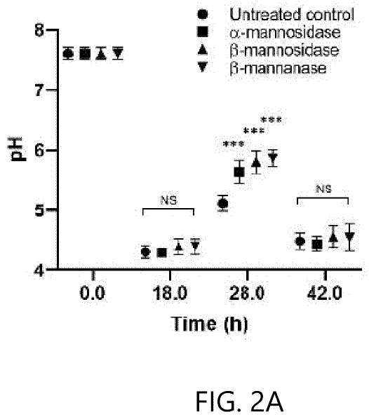

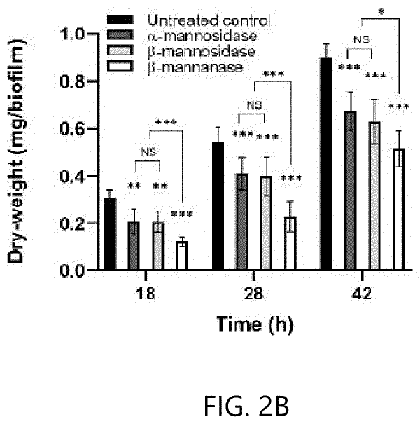

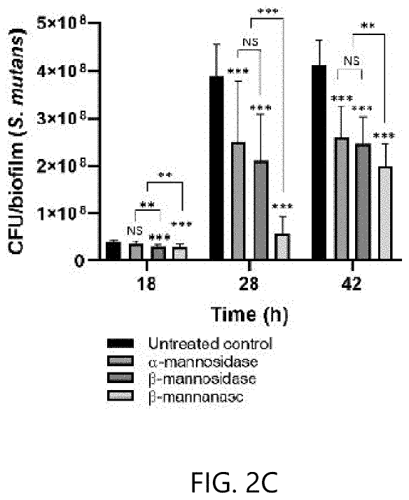

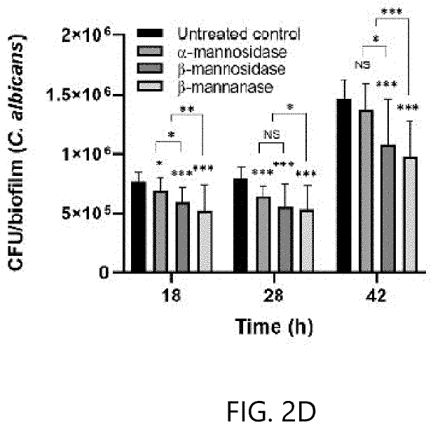

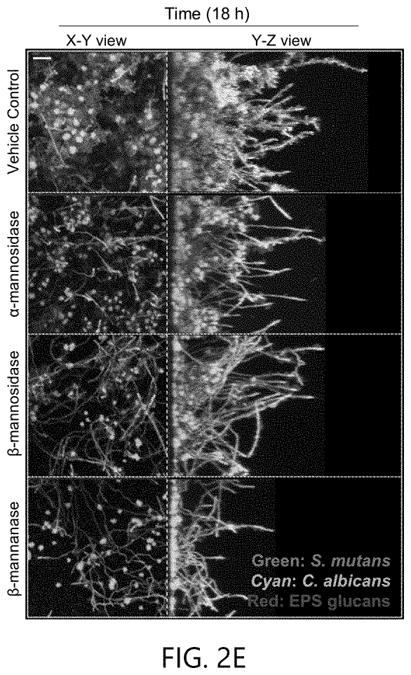

MDEs efficiently disrupt S. mutans-C. albicans biofilm development: the efficacy of the antibiofilm activity of MDEs was assessed using a well-established biofilm assay on hydroxyapatite discs. Biofilms were cultured in human saliva to more closely mimic the physiological condition. To assess the efficacy of a pre-determined dose of MDEs (e.g., 0.5, 0.2, or 10 U/well of α-mannosidase, β-mannosidase, or β-mannanase, respectively) on the cross-kingdom biofilm disruption, biofilms were evaluated following the regimen and comprehensively analyzed biofilm properties by measuring the pH of biofilm supernatant, dry-weight and CFU of biofilms (FIG. 2).

Salivary pH values under 5.5 are critical for tooth demineralization. For the untreated control, pH values remained below 5.5 throughout the biofilm experimental period, implying an acidic microenvironment conducive to tooth demineralization. At 28 h, in comparison to the untreated control's pH value of 5.08, the pH values rose close to pH 6 when treated with MDE (FIG. 2A). All three MDEs elevated the pH beyond the critical value of 5.5, signifying an alleviation of the acidic microenvironment.

The dry weight of biofilms was measured (FIG. 2B). There were significant reductions in the dry weights for all biofilms treated with MDEs in comparison to the untreated control. This trend was observed at all time points (18, 28, and 42 h). Overall, the MDEs led to a maximum reduction of dry weight at 28 h. The fold reductions in comparison to the untreated control were 2.5 for 13-mannanase and 1.4 for α- and β-mannosidase. This trend was also observed in the drops in CFU/biofilm (FIGS. 2C and 2D). The drops were greater for S. mutans than C. albicans. This can suggest that the loss of binding sites for GtfB on the cell walls of C. albicans prevented S. mutans from dense networking with C. albicans.

To further identify the differences in biofilm properties between samples treated with MDEs and the untreated control, the microbial growth and tertiary structures of the biofilms were assessed using confocal microscopy (FIG. 2E). Representative confocal images for 18 h biofilms depict a drastic drop in the amount of produced EPS, S. mutans-C. albicans mutualization and biofilm thickness. β-mannanase was most effective, followed by β-mannosidase and α-mannosidase.

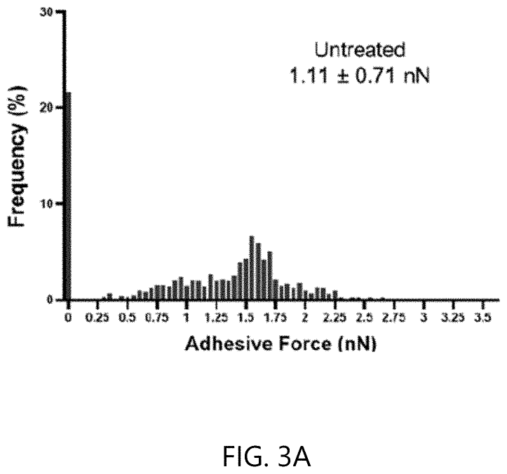

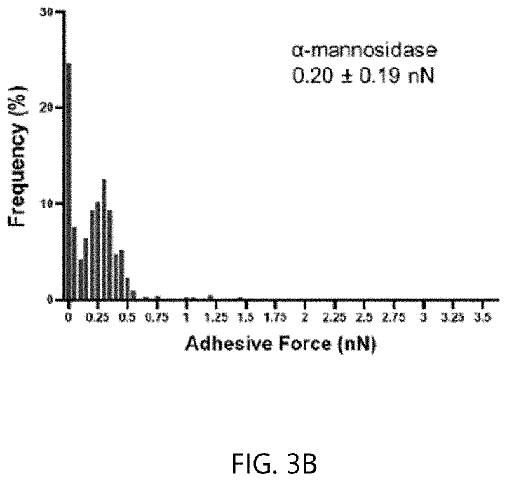

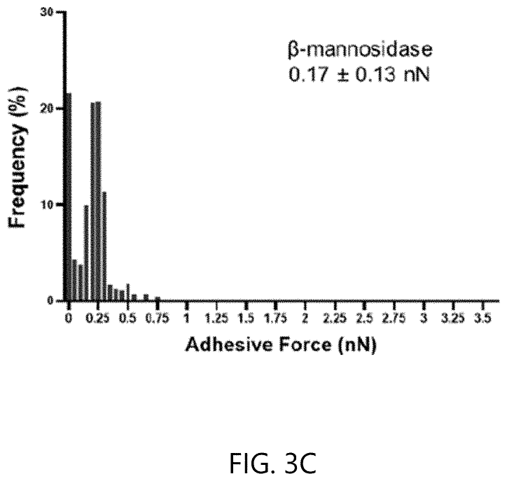

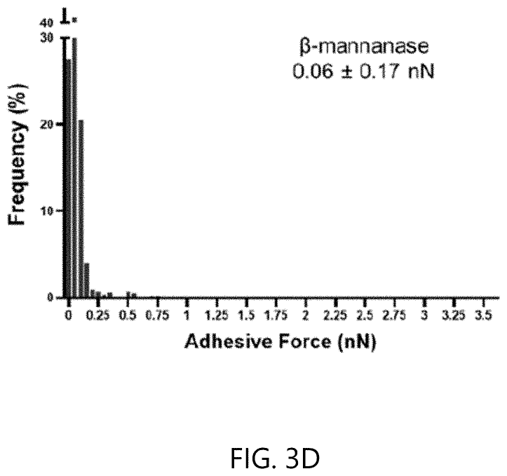

Reduced GtfB-C. albicans cell wall adhesion force for mannan-degraded C. albicans: GtfB binding strength to the surface of mannan-defective C. albicans was significantly reduced, which can be resulted in attenuated cross-kingdom biofilm development and tooth-demineralization in vivo. The use of MDEs can degrade the cell wall of C. albicans and limit biofilm interactions (FIGS. 1 and 2). The proposed mechanisms of S. mutans-C. albicans interaction were assessed via biophysical measurements of a reduction in GtfB-C. albicans binding forces for MDE-treated C. albicans. A dose-dependent reduction in GtfB-C. albicans binding forces were observed using single-molecule AFM (FIG. 3) following a similar methodology that used mannan-defective strains of C. albicans. Untreated C. albicans demonstrated strong binding forces of 1-2 nN towards GtfB (FIG. 3A). These forces were reduced up to 15-fold when C. albicans was treated with MDEs at optimal units for 5 min; a significant shift of GtfB binding distribution towards zero adhesive force was observed (FIGS. 3B-3D). These shifts significantly reduced the average binding forces of GtfB to the surface of α-mannosidase or β-mannosidase-treated C. albicans up to 5-fold (˜0.2 nN; FIGS. 3B, 3C). GtfB binding failure was almost doubled when C. albicans was treated with endoenzyme, β-mannanase, resulting in close to zero average binding force (0.06 nN, ˜15-fold reduction vs. untreated control; FIG. 3D). This data confirms the trend seen in assays to measure cell wall degradation and GtfB binding potential of MDE-treated C. albicans (FIG. 1) and the assays reporting antibiofilm effect against cross-kingdom biofilms (FIG. 2).

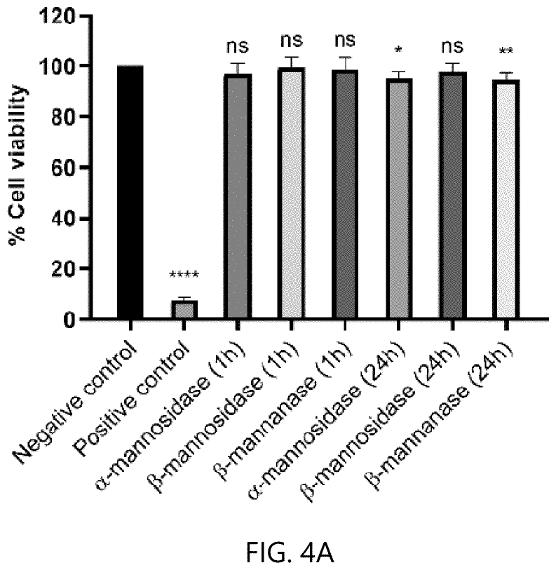

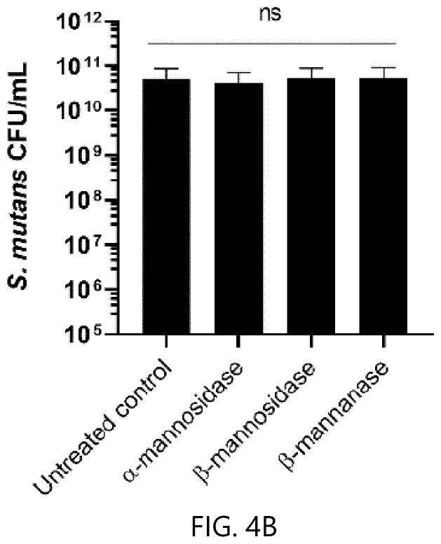

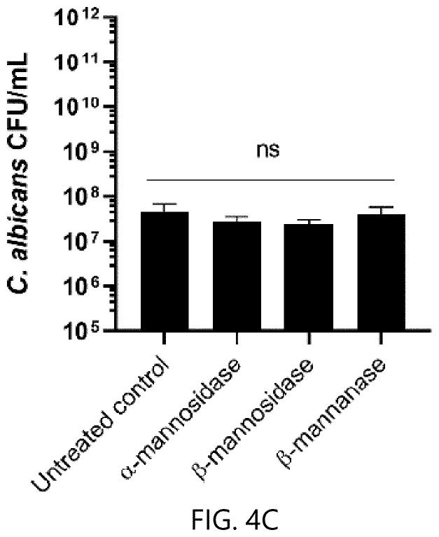

Cytotoxicity of MDEs against S. mutans, C. albicans, and human gingival keratinocytes: to be sustainable in biofilm disruption therapy, the proposed enzymatic treatment strategy needs to cause less antimicrobial resistance or toxic towards adjacent human cells in the oral cavity. Therefore, the microbicidal effect and cytotoxicity of the disclosed MDEs were evaluated. None of the disclosed MDEs exhibited a meaningful microbicidal effect; MDEs altered the growth kinetics of neither S. mutans nor C. albicans. Similarly, there was no discernible drop in CFU/mL for both S. mutans and C. albicans when they were exposed to different MDE units, including optimal units (FIGS. 4B and 4C). An MTT assay was performed on human gingival keratinocytes to depict the loss in % cell viability after exposure to MDEs at optimal units for 1 h and 24 h. An untreated group was included as a negative control and 3% H₂O₂-treated group as a positive control (where the keratinocytes cannot survive). The keratinocytes displayed no significant drop in cell viability (all >90%) when treated with any MDEs for either 1 h or 24 h exposure (FIG. 4A).

The disclosed non-microbicidal techniques targeting the receptor-ligand binding domain for cross-kingdom interactions using MDEs exhibited potency in suppressing S. mutans-C. albicans biofilm interactions by degrading the mannans on C. albicans cell wall without harming human gingival keratinocytes.

### Example 2

Early childhood caries (ECC), an aggressive form of tooth decay with rampant caries lesions, can be associated with frequent consumption of fermentable carbohydrates and poor oral hygiene. Early Childhood Caries can be defined as the presence of one or more decayed (e.g., non-cavitated or cavitated lesions), missing (due to caries), or filled tooth surfaces in any primary tooth in a preschool-age child between birth and 71 months of age. The microorganisms predominantly identified in ECC belong to Streptococci spp., Candida spp., Lactobacilli spp., Actinomyces spp., and Veillonella spp. Particularly, Candida albicans, an opportunistic fungal pathogen, is known to interact with cariogenic Streptococcus mutans to form biofilms associated with ECC. Symbiotic and synergistic interactions between these two kingdoms reinforce biofilm pathogenesis and the virulence of ECC.

Given the aggressive damage caused by ECC and its characterization as a polymicrobial disease with cross-kingdom consortia that develop hard-to-remove and highly acidic biofilms, there is a great need to strategically develop a targeted measure to effectively prevent cross-kingdom interactions and subsequent biofilm development. Certain endeavors to treat fungal-involved biofilm-associated diseases by using antibacterial or antifungal agents often exhibited limited efficacy due to a lack of targeting polymicrobial interactions. Furthermore, it is worth noting that these antimicrobials can disrupt ecological microbiota and/or induce drug resistance over time, providing significant limitations for preventive measures with long-term use. The cross-kingdom adhesion between S. mutans and C. albicans is dependent on the availability of sucrose and secreted bacterial exoenzymes (e.g., glucosyltransferases—Gtfs). Secreted Gtfs use sucrose to produce extracellular polymeric substances (EPS), in particular insoluble polysaccharides, which in turn form the extracellular matrix in cariogenic biofilms. GtfB from S. mutans can strongly bind to the C. albicans cell wall and leads to the enhanced production of EPS. Such elevated EPS amounts, in turn, lead to an increased number of binding sites for S. mutans, which promote their co-adhesion and subsequent biofilm formation in vivo. Furthermore, the mechanism of this biochemical interaction between GtfB and C. albicans, mannans on the C. albicans surface act as receptors for GtfB, thereby mediating the cross-kingdom interaction are involved. N- and O-linked mannan-defective mutant strains can exhibit severely reduced GtfB binding relative to wild-type strains, resulting in impaired maturation of cross-kingdom biofilms with S. mutans. These findings support development novel approaches targeting the adhesive interaction between S. mutans and C. albicans without necessarily being toxic to surrounding microbiota and tissues in the oral cavity.

Bolstered by the identification of the interkingdom receptor-ligand binding interaction, mannan-degrading enzymes (MDEs) can disrupt S. mutans-C. albicans interactions by reducing the number of binding sites available to form a mature cross-kingdom biofilm. Three MDEs (endoenzyme: 1,4-β-mannanase, and exoenzymes: α- and β-mannosidase) were used to disrupt S. mutans-C. albicans biofilm interactions as a targeted strategy to prevent ECC. The activity of MDEs was assessed in various buffers and human saliva. The ability of MDEs to degrade mannans on the C. albicans cell wall and to reduce the binding potential with GtfB was quantified. Then, the efficacy of MDEs to target S. mutans-C. albicans biofilms cultured on hydroxyapatite discs in human saliva to mimic physiological conditions in the oral cavity was determined. β-mannanase significantly diminished the cross-kingdom biofilm development, resulting in a ˜2.5-fold reduction of total biomass compared with the vehicle control. In addition, the mechanical stability of biofilms was remarkably weakened by β-mannanase treatment, causing near-complete surface detachment when exposed to low shear stress. Notably, the acidic environment induced by the cross-kingdom biofilms was alleviated, showing an elevated pH during biofilm development and reduced demineralization of the tooth enamel surface. To corroborate these results, single-molecule Atomic Force Microscopy (AFM) was used to measure GtfB-C. albicans binding forces. Data revealed a significant reduction in average binding forces for MDE-treated C. albicans (up to ˜15-fold reduction vs. vehicle control). MDEs were devoid of microbiocidal activity while showing no cytotoxicity against human gingival keratinocytes. Such a non-toxic but highly specific targeting of the interkingdom receptor-ligand binding interactions can lead to precision therapies for preventing biofilms associated with severe childhood dental caries.

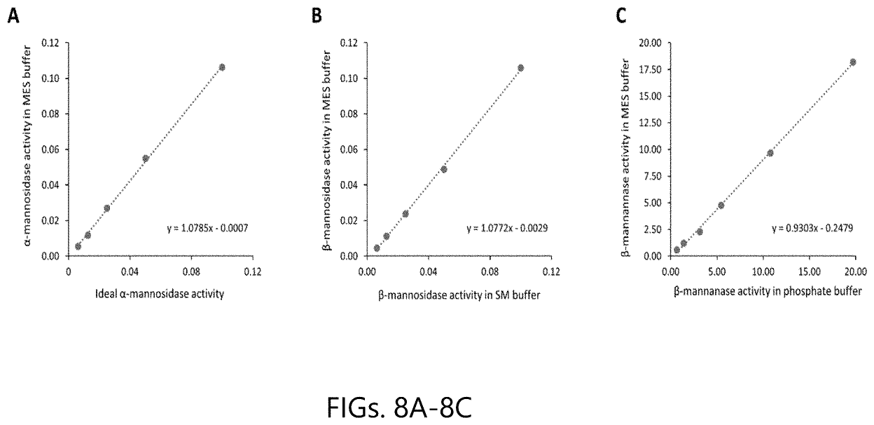

Enzyme activity in MES buffer and saliva: MDEs were chosen to degrade mannans on the cell wall of C. albicans and thereby reduce the incidence of the S. mutans-C. albicans biofilm interactions. As the efficacies of MDEs in cleaving mannans can be varied depending on their site of action, both exo- (α-mannosidase and β-mannosidase) and endo- (β-mannanase) mannan degrading enzymes were tested; the exoenzymes can hydrolyze terminal mannose residues while the endoenzyme can randomly hydrolyze mannosidic linkages within mannans. Before demonstrating their use, the MDEs were active against their respective substrates in various conditions and optimized the treatment time. The buffers for α-mannosidase, β-mannosidase, and β-mannanase can be IVIES, sodium maleate, and phosphate buffer, respectively. To ensure consistency during experiments and to reproducibly compare results, the activities of MDEs were compared in a single buffer (100 mM MES buffer with 2.5 mM CaCl₂ at pH 6.5 at 37° C.). As shown in FIG. 8, α-mannosidase showed 1.07, β-mannosidase showed 1.07, and β-mannanase showed 0.93 folds of enzyme activity in the MES buffer (vs. reported values from the manufacturers). It indicates that all the MDEs exhibited similar levels of enzyme activities when they were suspended in the IVIES buffer. Therefore, all the enzyme activities were measured at 5, 10, 30, and 60 min in 2-(N-morpholino) ethanesulfonic acid (MES) buffer to determine optimal condition.

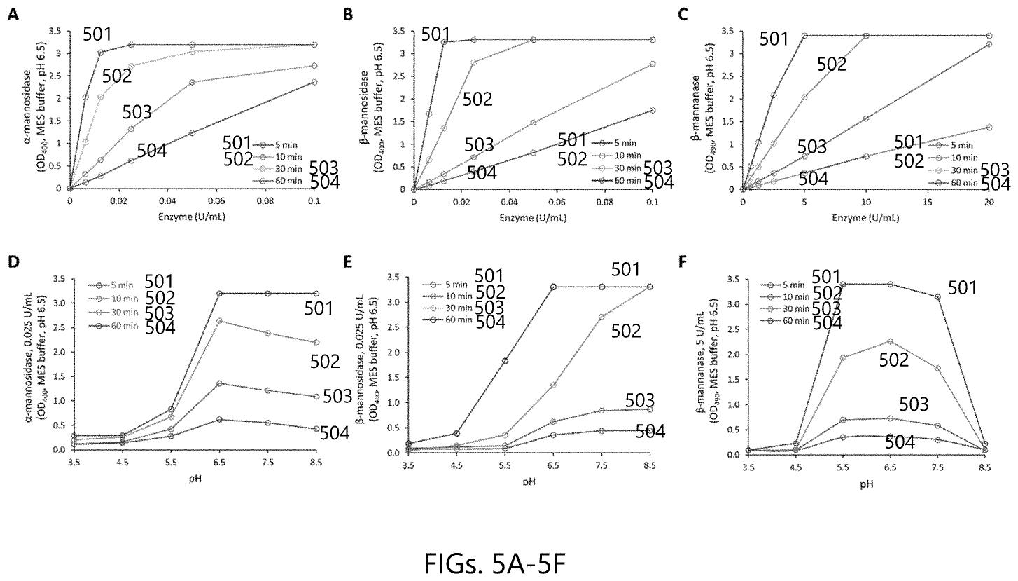

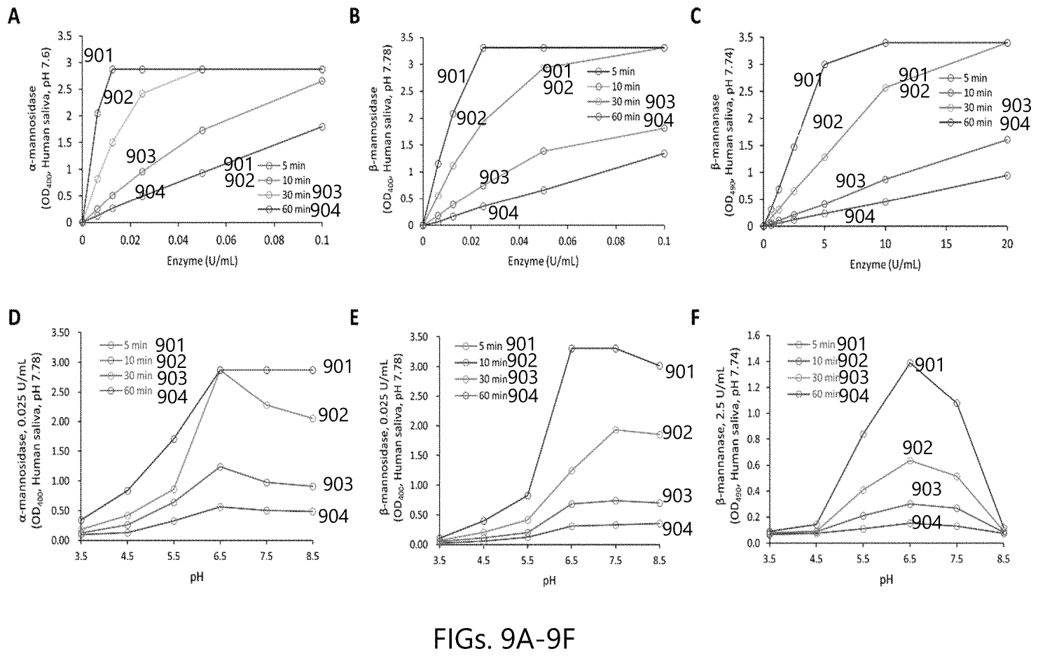

The activity profiles at different time points and pH values are depicted in FIG. 5. Enzyme activities were measured at different time points (e.g., 5 min 501, 10 min 502, 30 min 503, and 60 min 504) for (FIG. 5A) α-mannosidase, (FIG. 5B) β-mannosidase, and (FIG. 5C) β-mannanase. All MDEs had similar activity profiles for all time points. Activities of enzymes in acidic to neutral pH ranges were also determined for (FIG. 5D) α-mannosidase, (FIG. 5E) β-mannosidase, and (FIG. 5F) β-mannanase at different time points (e.g., 5 min, 10 min, 30 min, and 60 min). As shown, similar activity profiles were observed for all the tested conditions. The activities were saturated at higher units, and there was discernible activity at as early as 5 min for all MDEs (FIGS. 5A-5C). The pH profiles for α-mannosidase and β-mannosidase were similar; the highest activity was observed at and near pH 6.5 (FIGS. 5D-5E). For β-mannanase, the pH profile peaked near pH 6.5 but had a much sharper dip beyond pH 7.0 (FIG. 5F). Since the antibiofilm assays were conducted in human saliva, the activity profiles were measured with membrane-filtered saliva as the buffering system instead of MES buffer (FIG. 9).

FIG. 9 shows activity profiles for MDEs in saliva. Activities were measured at different time points (e.g., 5 min 901, 10 min 902, 30 min 903, and 60 min 904) for (FIG. 9A) α-mannosidase, (FIG. 9B) β-mannosidase, and (FIG. 9C) β-mannanase. All MDEs had similar activity profiles for all time points. pH profiles were measured for (FIG. 9D) α-mannosidase, (FIG. 9E) β-mannosidase, and (FIG. 9F) β-mannanase at different time points (e.g., 5 min 901, 10 min 902, 30 min 903, and 60 min 904). Results indicated similar profiles as FIG. 5 for all MDEs.

Degradation of C. albicans cell wall and GtfB binding potential: Since the MDEs displayed activity against their respective substrates within 5 min, and the optimal treatment time was determined as 5 min. After selecting an optimal treatment time, enzymatic cell wall degradation of C. albicans was demonstrated by calculating the glucose concentration in the supernatant and pellet (μg/mL) after treatment. FIGS. 1A-1C shows the effects of MDEs on the cell wall of C. albicans and its binding potential with GtfB. Dose-dependent degradation of the cell wall mannan (FIG. 1A) in the supernatant and (FIG. 1B) a corresponding decrease in the pellet of C. albicans, and (FIG. 1C) the amount of glucans formed on each C. albicans with or without MDEs treatment. The amount of mannans on MDE-treated C. albicans in supernatant increased while it decreased from the microbial pellet. In the presence of sucrose, lower amounts of bound GtfB in MDE-treated C. albicans led to reduced glucan formation. A dose-dependent increase in supernatant glucose concentration with increasing enzyme units for all MDEs (FIG. 1A). Consequently, there was a similar decrease in glucose concentration in the pellet, indicating reduced mannan components on MDE-treated C. albicans (FIG. 1B). From these results, the optimal enzyme units for cell-wall mannan degradation were 0.5, 0.2, and 10 U/well for α-mannosidase, β-mannosidase, and β-mannanase, respectively.

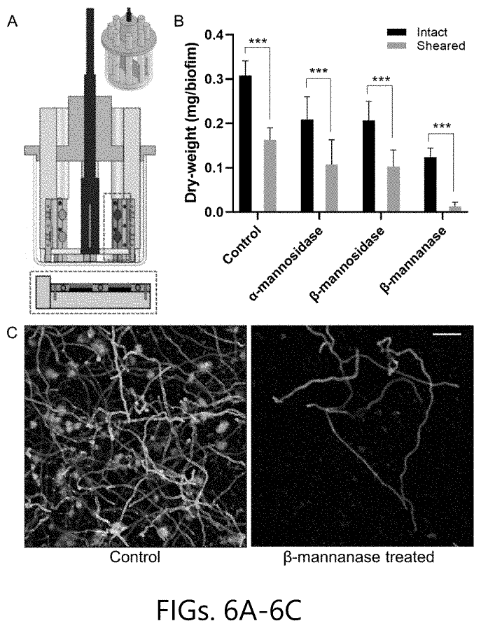

Mannans on the cell wall of C. albicans mediate GtfB binding to modulate S. mutans-C. albicans biofilm development. To demonstrate the effect of cell wall degradation on C. albicans to GtfB binding and activity by the use of MDEs, the binding potential of GtfB on the surface of C. albicans was determined. Each group of C. albicans with or without enzyme treatment was incubated with equal amounts of GtfB and sucrose to compare the amount of glucans formed on C. albicans. C. albicans treated with optimal units of MDEs for 5 min showed decreased glucan formation when compared to the vehicle control (FIG. 6C). Overall, β-mannanase was most effective (˜50% decrease in glucan formation) followed by β-mannosidase (˜35% decrease) and α-mannosidase (˜30% decrease). Results indicate that MDEs degraded the cell wall of C. albicans, and this led to fewer sites available for the binding of GtfB. Subsequently, in the presence of sucrose, this can lead to lower amounts of glucans formed.

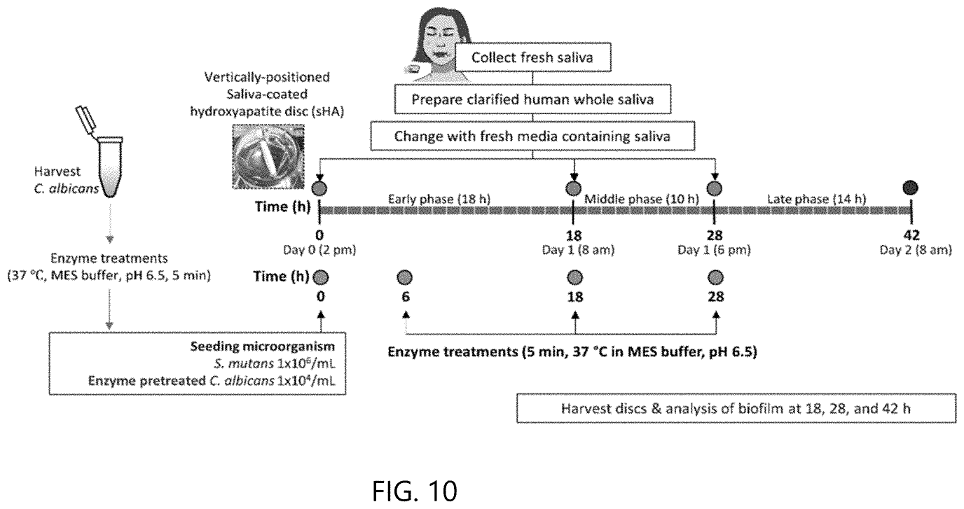

Disruption of S. mutans-C. albicans biofilm development: the efficacy of the antibiofilm activity of MDEs was assessed using a well-established biofilm assay on hydroxyapatite discs. Biofilms were cultured in human saliva to more closely mimic the physiological condition as depicted in FIG. 10. To assess the efficacy of a pre-determined dose of MDEs (e.g., 0.5, 0.2, or 10 U/well of α-mannosidase, β-mannosidase, or β-mannanase, respectively) on the cross-kingdom biofilm disruption, biofilms were assessed following the regimen and comprehensively analyzed biofilm properties by measuring the pH of biofilm supernatant, dry-weight and CFU of biofilms (FIG. 2). FIGS. 2A-2E shows the efficacy of MDEs against S. mutans-C. albicans biofilms: the pH of biofilm supernatant (FIG. 2A), dry weight per biofilm (FIG. 2B), CFU of S. mutans (FIG. 2C), and C. albicans (FIG. 2D) per biofilm. At optimal units, all MDEs had a significant antibiofilm effect on S. mutans-C. albicans biofilms as measured at 18, 28, and 42 h. Representative confocal images of untreated and MDE treated biofilms at 18 h. The scale bar indicates 20 μm (FIG. 2E).

Salivary pH values under 5.5 are critical for tooth demineralization. For the vehicle control, pH values remained below 5.5, implying an acidic microenvironment conducive to tooth demineralization. At 28 h, in comparison to the vehicle control's pH value of 5.08, the pH values rose close to pH 6 when treated with MDE (FIG. 2A). This is critical as all three MDEs elevated the pH beyond the critical value of 5.5, signifying an alleviation of the acidic microenvironment.

The dry weight of biofilms was measured (FIG. 2B). There were significant reductions in the dry weights for all biofilms treated with MDEs in comparison to the vehicle control. This trend was observed at all time points (18, 28, and 42 h). Overall, the MDEs led to a maximum reduction of dry weight at 28 h. The fold reductions in comparison to the vehicle control were 2.5 for β-mannanase and 1.4 for α- and β-mannosidase. This trend was also observed in the drops in CFU/biofilm (FIGS. 2C and 2D). The drops were greater for S. mutans than C. albicans. This suggests that the loss of binding sites for GtfB on the cell walls of C. albicans prevented S. mutans from dense networking with C. albicans.

The microbial growth and tertiary structures of the biofilms were assessed using confocal microscopy (FIG. 2E). Representative confocal images for 18 h biofilms depict a drastic drop in the amount of produced EPS, S. mutans-C. albicans mutualization and biofilm thickness. β-mannanase was most effective, followed by β-mannosidase and α-mannosidase. This result was confirmed with quantitative determinations of biovolume (μm³/μm²) for each channel (S. mutans, C. albicans, and EPS; FIG. 11). FIG. 11 shows the quantification of biovolumes for S. mutans, C. albicans and EPS with MDE treatment. Biovolumes (μm³/μm²) from confocal images for (FIG. 11A) S. mutans, (FIG. 11B) C. albicans, (FIG. 11C) EPS, and (FIG. 11D) total. All MDEs led to a reduction in the biovolume of S. mutans, C. albicans and EPS.

Effect of MDE treatment on the mechanical stability of biofilms: Disruption of S. mutans-C. albicans synergistic interaction can weaken the mechanical stability of biofilms. As shown, the amount of biomass and EPS were markedly altered (FIG. 2E) when biofilms were treated with MDEs. Large clumps detached from biofilms were observed after β-mannanase-treatment (data not shown). Thus, the function of the enzymatic strategy to facilitate biofilm removal was assessed using a custom-built device (FIG. 6A) that produces shear forces to detach biofilms from the sHA surface.

The ability of 18 h biofilms to withstand mechanical removal was assessed under shear stress by measuring the amount of biofilm that remained on the sHA before and after applying an estimated shear force (0.18 N/m²). The results showed that MDE-treated biofilms were more susceptible to surface detachment by shear force than vehicle control biofilms (FIG. 6B). This effect was more pronounced following β-mannanase-treatment, showing almost complete biofilm removal (˜90% vs. unsheared). Furthermore, representative confocal images of sheared biofilms showed that most of S. mutans microcolonies and C. albicans were detached from the disc surface when treated with β-mannanase, while untreated biofilms still contained numerous sizeable microcolonies and hyphal forms of C. albicans across the surface despite applied shear force (FIG. 6C).

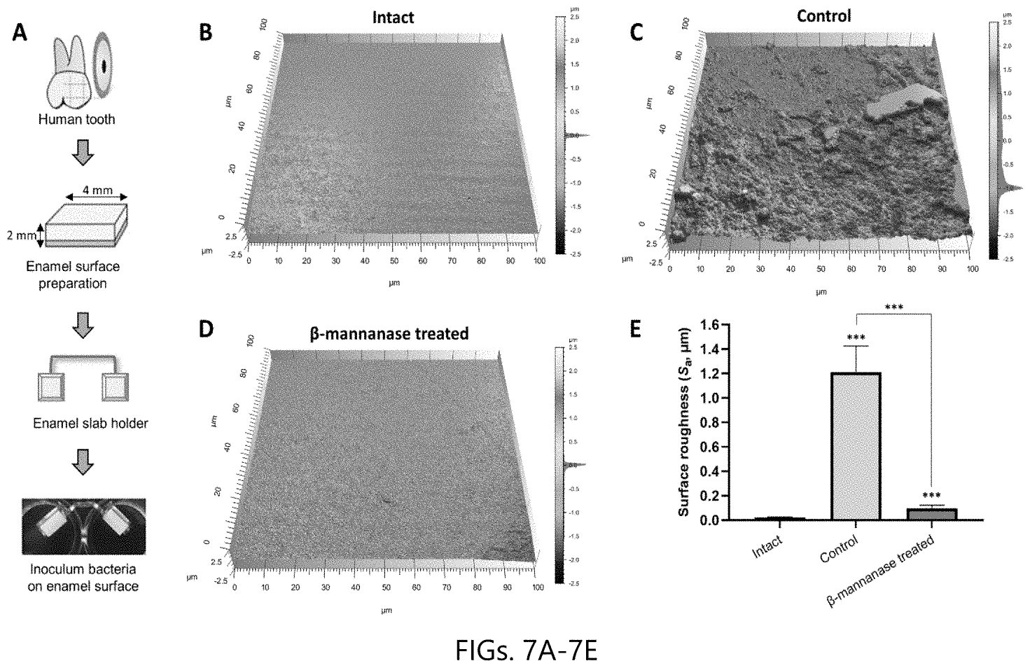

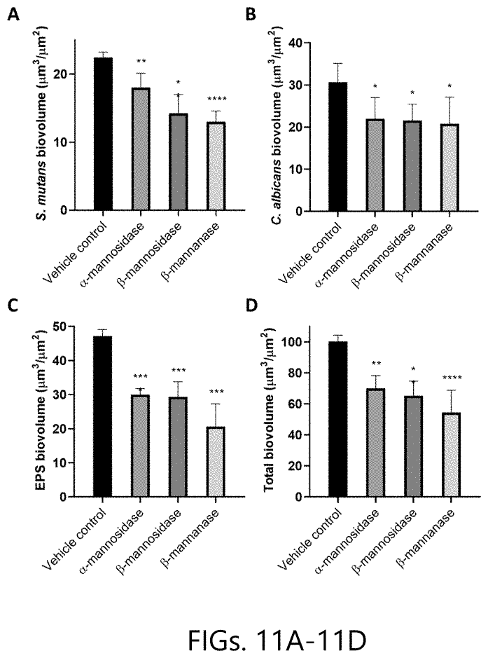

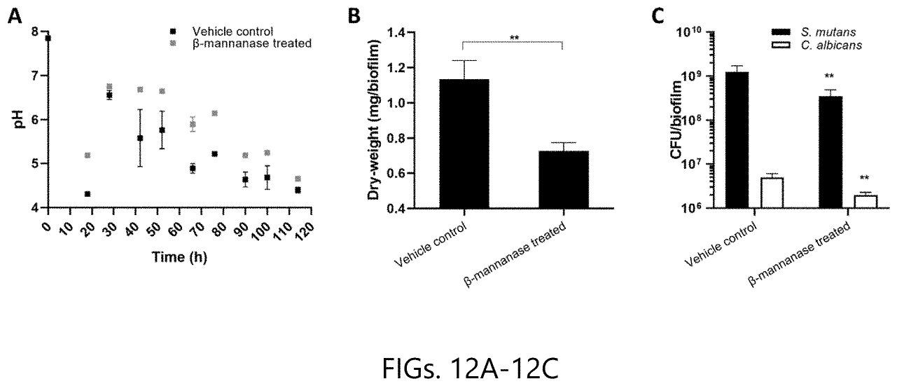

Effect of MDE treatment on the enamel surface demineralization: Reduced biofilm biomass and elevated pH by MDE treatment (FIG. 2) can also reduce tooth demineralization. Therefore, the level of enamel demineralization was investigated by culturing S. mutans-C. albicans biofilms (with or without β-mannanase treatment) on the human enamel slab (FIG. 7A) in saliva supplemented with 1% sucrose. By culturing biofilms for five days on human enamel slabs, similar patterns of pH, biofilm biomass, and CFU to the HA disc model (FIG. 12) were observed.

FIG. 12 shows the efficacy of MDEs against S. mutans-C. albicans biofilms on human enamel slab. The pH of biofilm supernatant (FIG. 12A), dry weight per biofilm (FIG. 12B), CFU of S. mutans (FIG. 12C) and C. albicans per biofilm. Then, the impact on enamel surface integrity was assessed by the treated biofilms both visually and quantitatively using confocal surface topographical analysis. A smooth and flat surface was observed from the intact surface prior to biofilm formation (FIG. 7B). However, the enamel surfaces underneath untreated S. mutans-C. albicans biofilms showed significantly eroded surfaces (FIG. 7C7C). In marked contrast, the mostly intact enamel surface was observed when S. mutans-C. albicans biofilms were treated with β-mannanase (FIG. 7D). This observation was supported by a quantitative analysis of arithmetical mean height (Sₐ) following ISO 25178. Overall, enamel surfaces were eroded by untreated S. mutans-C. albicans biofilms exhibited ˜13-fold higher Sₐ than those from β-mannanase treated S. mutans-C. albicans biofilms (FIG. 7E).

GtfB-C. albicans cell wall adhesion force for mannan-degraded C. albicans: GtfB binding strength to the surface of mannan-defective C. albicans was significantly reduced, which resulted in attenuated cross-kingdom biofilm development and tooth-demineralization in vivo. The use of MDEs can degrade the cell wall of C. albicans and thus limit biofilm interactions (FIGS. 1 and 2). Thus, the proposed mechanisms of S. mutans-C. albicans interaction was assessed via biophysical measurements of GtfB-C. albicans binding forces for MDE-treated C. albicans using single-molecule AFM. A dose-dependent reduction in GtfB-C. albicans binding forces was observed (FIG. 3), following a similar pattern to that found in mannan-defective strains of C. albicans. Untreated C. albicans demonstrated strong binding forces of 1-2 nN towards GtfB (FIG. 3A). These forces were significantly reduced when C. albicans was treated with MDEs at optimal units for 5 min; a drastic shift of GtfB binding distribution towards zero adhesive force was observed (FIGS. 3B-3D). These shifts significantly reduced the average binding forces of GtfB to the surface of α-mannosidase or β-mannosidase-treated C. albicans up to 5-fold (˜0.2 nN; FIGS. 3B and 3C). GtfB binding failure was almost doubled when C. albicans was treated with endoenzyme, β-mannanase, resulting in close to zero average binding force (0.06 nN, ˜15-fold reduction vs. vehicle control; FIG. 3D). This data confirms the trend found in bioassays to measure cell wall degradation and GtfB binding potential of MDE-treated C. albicans (FIG. 6) and the antibiofilm effect against cross-kingdom biofilms (FIG. 2).

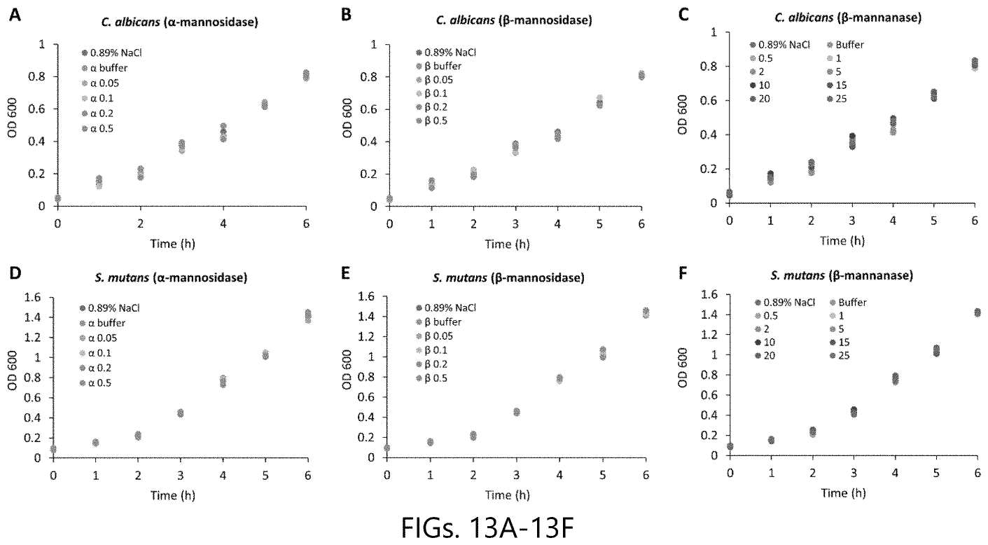

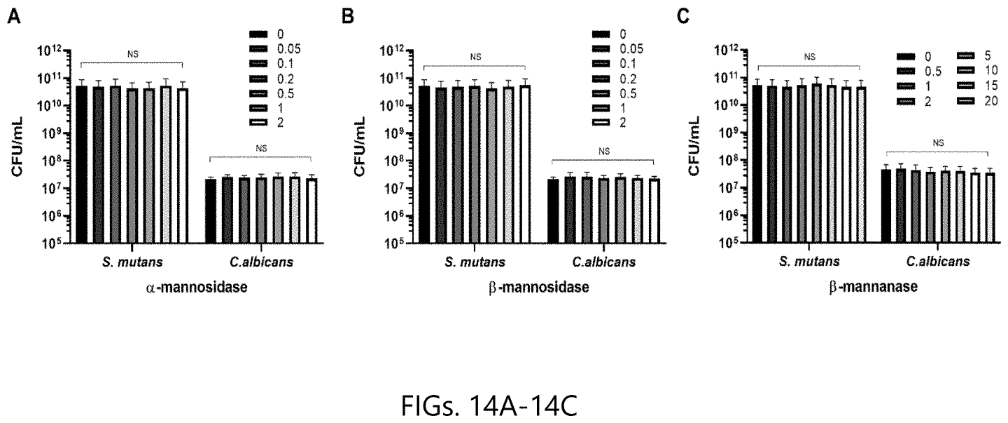

Cytotoxicity of MDEs against human gingival keratinocytes: For the proposed enzymatic treatment strategy to be sustainable in biofilm disruption therapy, antimicrobial resistance and toxicity toward adjacent human cells in the oral cavity needs to be decreased. Therefore, the microbicidal effect and cytotoxicity of the disclosed MDEs were evaluated. None of the disclosed MDEs exhibited a meaningful microbicidal effect; MDEs altered the growth kinetics of neither S. mutans nor C. albicans (FIG. 13). FIGS. 13A-13F shows the growth kinetics of S. mutans and C. albicans after treatment with MDEs. Growth curves for C. albicans after treatment with α-mannosidase (FIG. 13A), β-mannosidase (FIG. 13B), and β-mannanase (FIG. 13C). Growth curves for S. mutans after treatment with α-mannosidase (FIG. 13D), β-mannosidase (FIG. 13E), and β-mannanase (FIG. 13F). All MDEs did not affect the growth curves of both microorganisms. Similarly, there was no discernible drop in CFU/mL for both S. mutans and C. albicans when they were exposed to different MDE units, including optimal units (FIGS. 4B, 4C, and 14). An MTT assay was performed on human gingival keratinocytes to depict the loss in % cell viability after exposure to MDEs at optimal units for 1 h and 24 h. The vehicle group was included as a negative control, and 3% H₂O₂-treated group was included as a positive control (where the keratinocytes cannot survive). The keratinocytes displayed no significant drop in cell viability (all >90%) when treated with any MDEs for either 1 h or 24 h exposure (FIG. 4A). Collectively, a non-microbicidal tactic targeting the receptor-ligand binding domain for cross-kingdom interactions using MDEs exhibited great potency in suppressing S. mutans-C. albicans biofilm interactions by degrading the mannans on C. albicans cell wall without displaying microbiocidal effects or harming human gingival keratinocytes.

Given its prevalence across all demographic and social variables, ECC can pose a public health issue in both developing and industrialized countries. Among the various factors affecting ECC development, heavy infection by S. mutans and C. albicans under a sugar-rich diet has been shown to be an important microbiological feature in severe ECC.

An enzymatic approach that can specifically degrade mannans on the C. albicans cell wall to interrupt S. mutans-C. albicans biofilm interactions using biophysical, biochemical, and microbiological methods were evaluated. MDEs effectively degraded mannans on C. albicans (FIGS. 1A and 1B), disrupted GtfB binding to C. albicans (FIG. 1C), and attenuated S. mutans-C. albicans biofilm development and acidogenicity (FIG. 2). Dose-dependent degradation of the C. albicans cell wall was accompanied by an increased reduction of GtfB binding and subsequent disruption of localized glucan production. Overall, β-mannanase was significantly (up to 2.5-fold) more effective than β-mannosidase and α-mannosidase in exerting antibiofilm activity. This included a significant reduction in total biofilm biomass as well as the content of individual biofilm components (FIGS. 2 and 11). Furthermore, biofilms treated with β-mannanase inflicted minimal tooth-enamel surface demineralization (FIG. 7).

The antibiofilm mechanism of this approach was further assessed using biophysical methods. It has been observed that GtfB-C. albicans binding forces were significantly lower for mannan-defective mutant C. albicans in comparison to the wild type. Binding forces of GtfB to β- or N-mannan mutant strains ranged from ˜0.2 nN to ˜0.5 nN, which were several-fold less, compared with wild type (1-2 nN). Thus, the binding forces of GtfB-C. albicans cell-wall surface were determined using single-molecule AFM to confirm whether disruption of GtfB-to-mannan binding is the driving mechanism for MDE antibiofilm activity. The endoenzyme β-mannanase treatment of C. albicans reduced the binding forces of GtfB-to-C. albicans by ˜15-fold and increased GtfB binding failure by ˜2-fold (vs. the vehicle control; FIG. 3D). These binding force values were comparable to the values for N-mannan mutant strain och1ΔΔ. Likewise, treatment of C. albicans with either exoenzyme, α- or β-mannosidase, led to significant reductions in the GtfB binding forces (˜5-fold) vs. the vehicle control. These binding force values were similar to those for β-mannan mutant strains pmt1ΔΔ or pmt4ΔΔ, demonstrating the efficacy of MDE to target the mannan structure on the fungal surface.

The observed differences in MDE efficiencies against cross-kingdom biofilm and GtfB binding can occur possibly due to the cleavage characteristics of the MDEs. Since β-mannanase as an endoenzyme can randomly hydrolyze internal/intramolecular mannosidic linkages, it can induce the detachment of bulky mannans from C. albicans. In contrast, exoenzymes, α-mannosidase, and β-mannosidase can only degrade terminal linkages to liberate residues gradually, resulting in reduced removal of mannans (vs. β-mannanase). Such effects at the single-cell level can also affect the mechanical properties of the biofilm as a whole. Using a fluid shear-inducing device, a significant reduction was observed in the mechanical strength of β-mannanase-treated biofilms, increasing surface detachment under low shear stress (FIG. 6). This indicates that the MDE strategy can also compromise the biofilm bulk stability, facilitating biofilm removal from the apatitic surface. Collectively, the results show that disruption of the GtfB-mannan interactive ligand-receptor domain effectively impairs the interkingdom co-adhesion mechanism while also affecting the biofilm mechanical integrity.

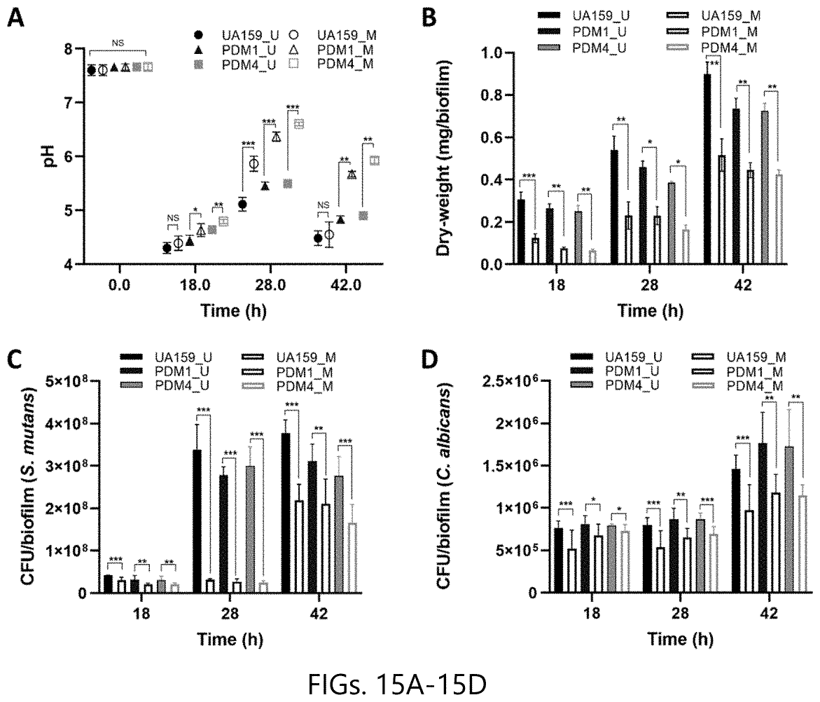

Receptor-specific targeting of the surface of C. albicans can be critical for the successful intervention of GtfB-C. albicans interaction using MDE. Although both fungal and mammalian cells glycosylate proteins via similar mechanisms, a key difference is that N-linked and O-linked glycans on fungal (but not in mammalian) proteins are predominately composed of mannose. Since MDEs exhibit high specificity to mannose, it is likely that MDEs preferably bind to and hydrolyze mannose on fungal cells. However, there are other glycoproteins in saliva, and whether the efficacy of MDEs can be affected by other potential competitive substrates in the oral cavity can be assessed. The disclosed MDE approach can work with clinical isolates of S. mutans from ECC plaque that can have distinctive phenotype and biological properties. β-mannanase treatment was equally effective against biofilms formed with S. mutans clinical isolates (PDM1 and PDM4) compared to the ones with S. mutans UA159 (FIG. 15). FIGS. 15A-15D shows the efficacy of MDEs against S. mutans-C. albicans biofilms formed with reference (UA159) strain or clinical isolates (PDM1 or PDM4) of S. mutans: (FIG. 15A) the pH of biofilm supernatant, (FIG. 15B) dry weight per biofilm, CFU of (FIG. 15C) S. mutans, and (FIG. 15D) C. albicans per biofilm. At optimal enzyme units, all MDEs had a significant antibiofilm effect on S. mutans-C. albicans biofilms as measured at 18, 28, and 42 h.

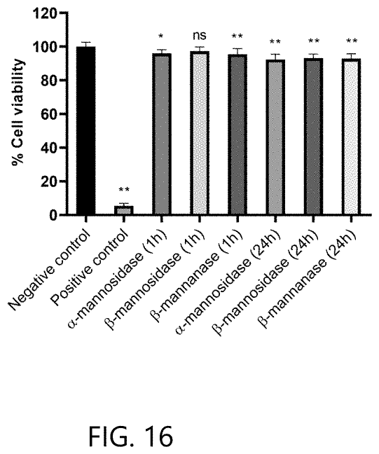

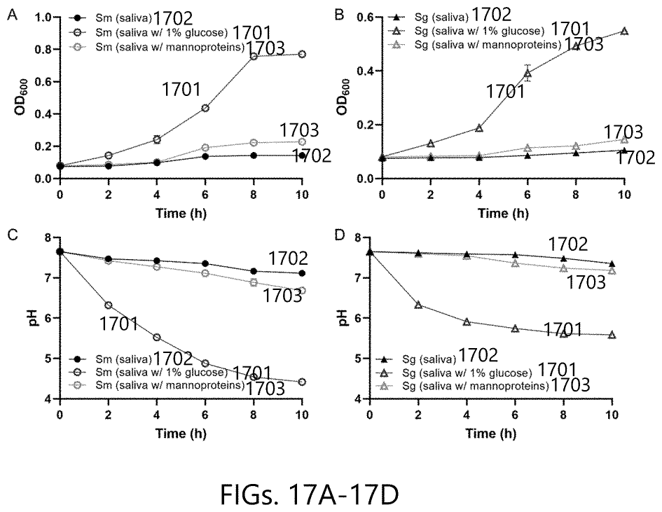

In addition, enzyme stability in the oral environment can be equally important for therapeutic activity. Notably, MDEs maintained their enzymatic activities under physiologically relevant conditions (in complex human saliva; FIGS. 2 and 9). Results also show that MDEs were relatively stable under a non-optimal buffer solution (MES buffer; FIG. 8) while maintaining catalytic activity across pH variations during biofilm grows, suggesting that the enzymes stay active under various surrounding environments. Despite their enzymatic stability, MDEs did not interfere with the growth and viability of S. mutans and C. albicans (FIGS. 13 and 14), which can avoid the development of antimicrobial resistance over time. Moreover, the lack of cytotoxicity of MDEs towards human gingival keratinocytes (FIG. 4), in addition to preventive effects against tooth-enamel demineralization, augurs well for its targeting specificity and potential clinical applications as a therapeutic agent. MDEs at a 5-fold higher concentration than the optimal unit did not induce severe cellular inflammation (FIG. 16), which mitigates concern on the potentially deleterious effects of MDEs accumulation in the oral cavity. FIG. 16 shows the toxicity assay of 5-fold of the optimal unit of MDEs on human gingival keratinocytes. Normalized cell viability for HGKs after exposure to 5-fold of the optimal units of MDEs for 1 h and 24 h was shown. No significant loss in HGK cell viability was observed for 5×MDE treatments. Negative control and positive control represent vehicle control and 3% H₂O₂ control, respectively.

Since MDEs hydrolyze mannose from C. albicans surface, it is possible that cleaved mannoproteins can be utilized for bacterial growth and/or metabolic activity as reported elsewhere. However, the estimated amount of released mannoproteins from C. albicans is extremely low (˜500-fold less) compared with the supplemented carbon source (i.e., 1% sucrose or glucose). To test this, mannoproteins were extracted from C. albicans by β-mannanase and utilized to compare the growth of S. mutans and Streptococcus gordonii and respective pH changes. As expected, significant growth of either microorganism with limited pH drop was not observed when cultured in saliva supplemented with extracted mannoproteins. In contrast, those cultured in saliva with 1% glucose 1701 showed exponential growth and significant reduction of pH over time (FIGS. 17A-17D). FIG. 17 shows the growth of S. mutans and S. gordonii and pH changes over time. Bacteria cultured in saliva supplemented with glucose 1701 showed exponential growth of bacteria and logarithmic reduction of pH over time. Bacteria cultured in saliva only 1702 or saliva supplemented with extracted mannoproteins 1703 from C. albicans via β-mannanase treatment were devoid of major effects.

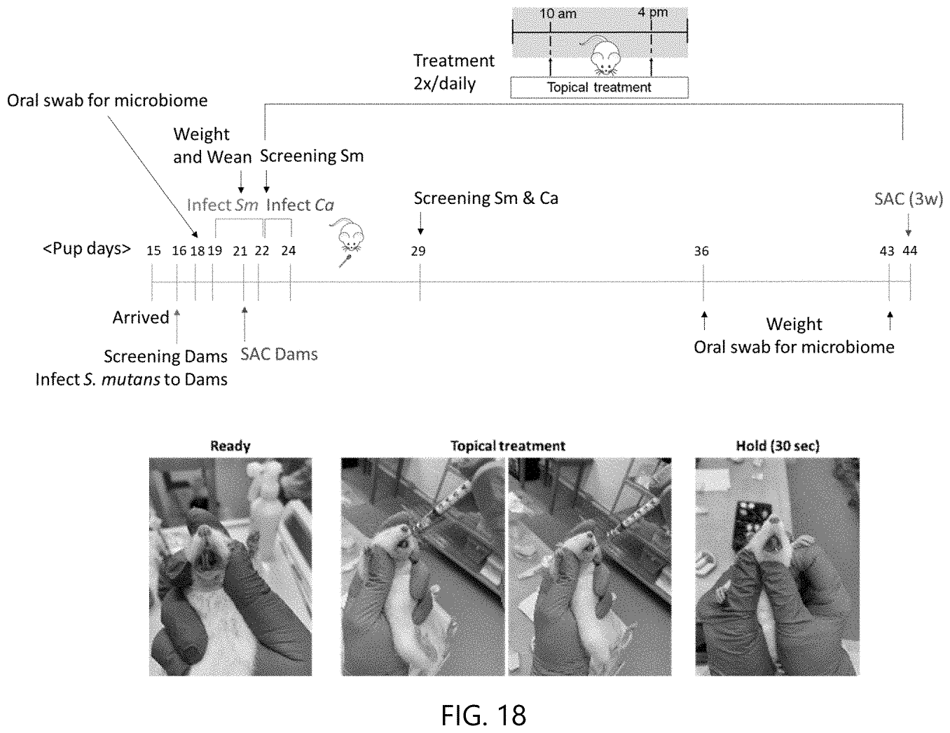

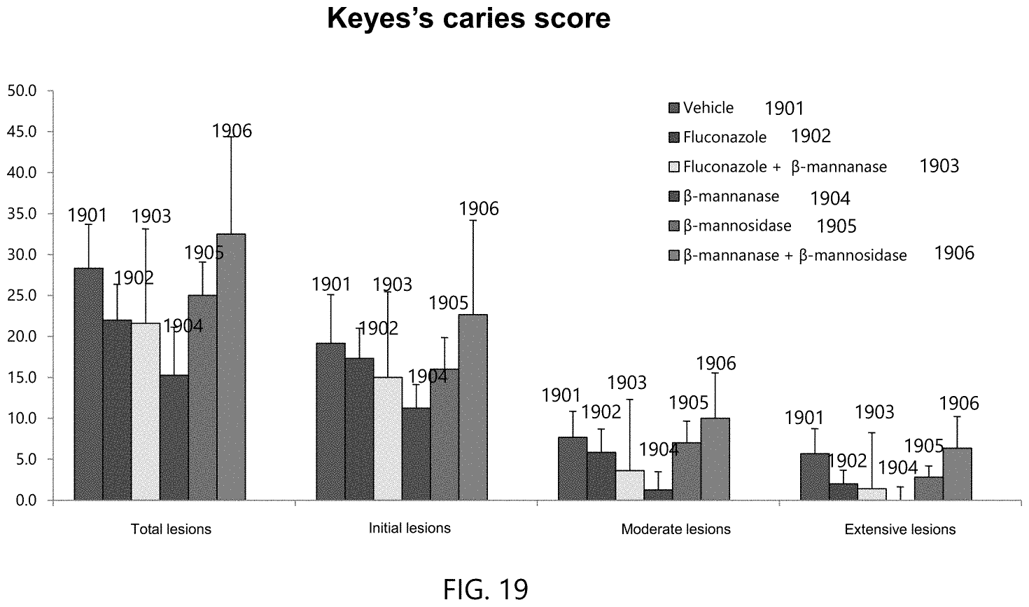

To determine the impact of a topical MDEs treatment on tooth decay, a rodent model, which mimics the characteristics of severe early childhood caries that includes S. mutans infection of rat pups and protracted feeding of a sugar-rich diet, was used. Conditions that can be experienced clinically in humans were considered using the rodent model by applying the test agent solutions topically (orally delivered; 100 μl per animal) twice daily with a brief, 30 sec exposure time (FIG. 18) to mimic the use of mouthwash.

Using this treatment regimen, the incidence and severity of caries lesions on the teeth of rat pups were assessed. During the 3-week period, the rats remained in apparent good health, and no significant differences in body weights between control and all test groups were detected. Treatments with a unit of β-mannanase resulted in potent suppression of caries development at all relevant sites (both smooth and sulcal surfaces). As shown in FIG. 19, quantitative caries scoring analyses showed that a unit of β-mannanase greatly attenuated the initiation and severity of caries lesions (vs. vehicle control, FIG. 19, P<0.05 by one way ANOVA with post hoc Tukey HSD test), and completely blocked extensive enamel damage, preventing the onset of cavitation on both smooth and sulcal dental surfaces. Furthermore, the efficacy of β-mannanase was significantly higher than 0.2% Fluconazole (P<0.05 by one-way ANOVA with post hoc Tukey HSD test), reducing more effectively the number and severity of caries lesions. Proportionally greater effects on moderate and extensive carious lesions than on initial caries were observed, which can be related to the conditions mimicking severe childhood caries. Considering the dynamics of caries development, the effects on less severe lesions can be observed at earlier time points.

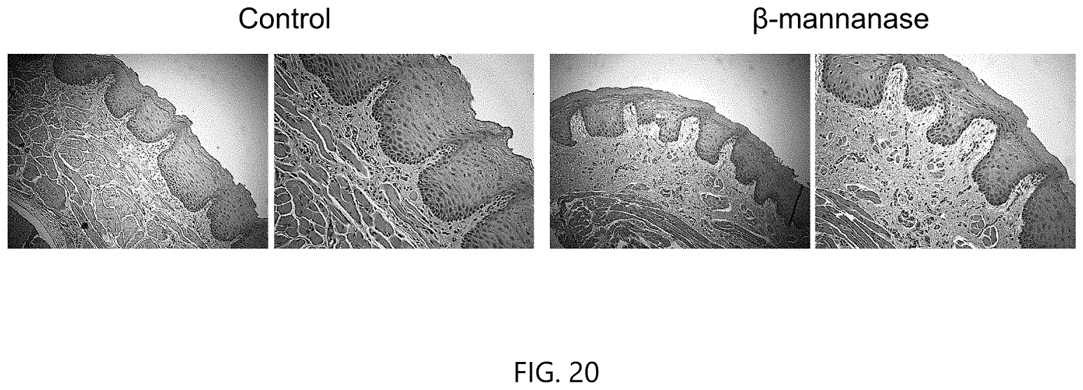

To evaluate the overall effects on surrounding tissues after 21 days of topical treatment, the histopathological images of soft oral tissues are presented in FIG. 20. Hematoxylin and eosin images of gingival tissues showed that both vehicle control and β-mannanase treated groups had no visible signs of harmful effects such as proliferative changes, inflammatory responses, or necrosis, indicating high histocompatibility of MDEs treatment. Taken together, the data show that topical MDE treatments can efficiently suppress the development of a prevalent oral disease without showing deleterious effects in the surrounding soft tissues in vivo.

Target specificity and retention of antibiofilm agents, as well as their penetration behaviors into the biofilm, can determine the fate of the antibiofilm strategy. For example, enhanced retention of antibacterial agent-loaded nanoparticles resulted in a dramatic improvement in antibiofilm activity compared with the non-loaded antibacterial agent. Thus, enhanced retention and penetration of MDE can further improve the efficacy of this approach. Phagosome maturation can be enhanced for C. albicans O-mannosylation mutant (defective in cell wall mannans) due to exposure of β-glucan in the inner cell wall. This finding indicates that MDEs can mitigate cellular inflammation caused by fungal-mediated infections. In vivo studies can provide further insights into this additional therapeutic effect.

The results revealed that targeting and intervening in the interkingdom receptor-ligand binding interactions using MDEs can lead to a novel, non-biocidal and more precise therapeutic measure. The enzymes are stable in complex human saliva and enzymatically active within a biofilm environment, efficiently degrading mannans on C. albicans cell wall and, in turn, significantly impairing its binding potential with GtfB. The targeted disruption of receptor-ligand at the cellular level inflicted changes at the macroscale affecting biofilm biomass, population, mechanical stability, and acidity, culminating with a marked reduction of human tooth-enamel demineralization. These properties were achieved without microbiocidal effects or causing cytotoxicity to human cells, suggesting a potential application as a targeted approach for disrupting a pathogenic cross-kingdom biofilm associated with severe ECC, a costly and unresolved oral infectious disease.

Strains and culture conditions” Candida albicans SC5314, a well-characterized fungal strain, and Streptococcus mutans UA159, a proven virulent cariogenic dental pathogen and well-characterized EPS producer, were used for biofilm experiments. Microbial stocks were stored at ˜80° C. in tryptic soy broth containing 50% glycerol before use. All strains were grown to mid-exponential phase (optical densities at 600 nm of 0.8 (C. albicans) and 1.0 (S. mutans), respectively) in ultrafiltered (10 kDa molecular-mass cutoff; Millipore, Billerica, Mass., USA) yeast—tryptone extract broth containing 2.5% tryptone and 1.5% yeast extract (UFYTE; pH 5.5 and 7.0 for C. albicans and S. mutans, respectively) with 1% (wt/vol) glucose at 37° C. and 5% CO₂ as described previously. Cells were harvested by centrifugation (6,000 g, 10 min, 4° C.).

Mannan Degrading Enzymes (MDEs): Purified exo-α-mannosidase (EC 3.2.1.24) was purchased from Sigma (MO, USA). Purified exo-β-mannosidase (EC 3.2.1.25) was purchased from Megazyme (Bray, Ireland). Purified endo-β-mannanase (EC 3.2.1.78) was purchased from Megazyme (Bray, Ireland). A unit of α-mannosidase activity is defined as the amount of enzyme required 1 μmole of p-nitrophenol (pNP) per min from p-nitrophenyl-α-D-mannopyranoside (5 mM) in IVIES buffer (100 mM) and CaCl₂ (2.5 mM) at pH 6.5 at 40° C. A unit of β-mannosidase activity is defined as the amount of enzyme required to release 1 μmole of pNP per min from p-nitrophenyl-β-D-mannopyranoside (0.8 mM) in sodium maleate buffer (100 mM) at pH 6.5 at 35° C., monitored at 400 nm. A unit of β-mannanase activity is defined as the amount of enzyme required to release 1 μmole of mannose reducing-sugar equivalents per minute from carob galactomannan in sodium phosphate buffer (100 mM), pH 7.0 at 40° C.

Saliva collection: Written informed consent was obtained from all volunteers. Saliva was collected from healthy donors who had not taken any medications for at least a month. The donors chewed unflavored paraffin wax, and saliva was collected in a conical tube on ice. Saliva was collected in the morning without having breakfast. Collected saliva was centrifuged (5,500 g, 4° C., 10 min), followed by filter sterilization (0.22 μm; S2GPU01RE ultra-low binding protein filter; Millipore, Billerica, Mass.). Filtered saliva was then kept at 4° C. until use.

C. albicans cell wall degradation assay: C. albicans were grown to mid-exponential phase (optical densities at 600 nm of 0.8) in UFYTE, pH 5.5 containing 1% (wt/vol) glucose. An aliquot (1 mL) of the cell suspension was centrifuged at 10,000 g for 10 min at 4° C. The cell pellet was resuspended and washed in the same volume of 1×PBS buffer (Dulbecco's Phosphate-Buffered Saline, 1×, Corning Inc., Corning, N.Y., USA) with a pH of 7.33. This procedure was repeated twice to remove any remaining sugar. After treatment with MDEs (IVIES buffer, pH 6.5, 37° C., 5 min), the supernatant was collected, and the cell pellet was resuspended in the same volume of IVIES buffer. All the supernatants were pooled, three volumes of cold ethanol were added, and the resulting precipitate was collected and resuspended in water. These precipitates were polysaccharides released from the cell wall after enzymatic treatments. Mannans from pellets were isolated using a mild alkali extraction method with boiling for 60 min. Harvested pellets were washed with 1×PBS and then resuspended in 2% (w/v) KOH. This suspension was boiled for 60 minutes to extract mannan. The amount of reducing sugars was determined by the Somogyi-Nelson colorimetric assay.

Estimation of GtfB binding potential: An overnight culture of C. albicans was subcultured to an OD of 0.8. The subculture was centrifuged (5,500 g, 4° C., 10 min) followed by a wash with 1×PBS to remove all the nutrient media and resuspended in 3 mL of MES buffer (prewarmed at 37° C.). The suspension was split into 0.5 mL aliquots, and respective MDEs were added at optimal units for 5 minutes (incubate at 37° C.). Samples were then spun down and washed with 1×PBS to remove all the enzymes. The pellets were resuspended in 0.4 mL of adsorption buffer and incubated with 25 μg/mL of GtfB for 30 min at 37° C. Samples were then spun down and washed with 1×PBS to remove all the GtfB. Next, the pellets were resuspended in 0.5 mL of sucrose substrate for 1 h at 37° C. Samples were then spun down and washed with 1×PBS. The pellets and formed glucans were resuspended in 1 mL of 1N NaOH. Lastly, glucans formed were estimated colorimetrically.

In vitro biofilm model: Biofilms were formed using our saliva-coated hydroxyapatite (sHA) model. For HA disc (surface area, 2.7±0.2 cm2; Clarkson Chromatography Products, Inc., South Williamsport, Pa.) coating, saliva was mixed with adsorption buffer at 1:1 ratio and clarified by centrifugation followed by filter sterilization as described previously. The HA discs were vertically suspended in 24-well plates using a custom-made wire disc holder, mimicking the free smooth surfaces of the pellicle-coated teeth. C. albicans were pretreated with each MDE for 5 min before inoculation. Each disc was inoculated with approximately 2×10⁶ CFU of S. mutans/ml and 2×10⁴ CFU of C. albicans/ml in prepared filter-sterilized saliva supplemented with 1% (w/v) sucrose at 37° C. under 5% CO₂. The proportion of the microorganisms in the inoculum is similar to that found in plaque samples from children with ECC.

As illustrated in FIG. 10, the discs were treated with MDEs 3 times (6, 18, and 28 h) during biofilm formation. For enzyme treatment, each disc with biofilm was transferred to the pre-warmed IVIES buffer (37° C.) containing each enzyme, incubated for 5 min, and then transferred back to the cultured medium (6 h) or fresh medium (18 h and 28 h). For the vehicle control, each disc with biofilm was transferred to the pre-warmed MES buffer not containing MDE (37° C.), incubated for 5 min, and then transferred back to the cultured medium (6 h) or fresh medium (18 h and 28 h). The culture medium was changed twice daily at 8 am and 6 pm, and the pH of the supernatant was determined using an Orion pH electrode attached to an Orion DUAL STAR™ pH meter (Thermo Fischer Scientific, Waltham, Mass., USA) until the end of the experimental period (42 h). The biofilms were collected at 18 h, 28 h, and 42 h for imaging and biochemical analysis.

In parallel, biofilms were also formed with two clinical isolates from plaque samples collected from ECC children, and the efficacy of MDE treatment was evaluated to further determine the feasibility of the clinical application. These clinical isolates of S. mutans were identified using Mitis Salivarius Agar plus Bacitracin (MSB) agar plates. All the biofilm experiments were performed following the procedures described above.

Microbiological and biochemical biofilm analysis: Collected biofilms at each time point were subjected to standard microbiological and biochemical analysis. Briefly, the biofilms were removed and homogenized by sonication, and the number of viable cells (CFU/biofilm) was determined. In parallel, an aliquot of biofilm suspension was centrifuged (5,500 g, 10 min, 4° C.), and the pellet was washed twice with Milli-Q water, dried in an oven (105° C., 24 h), and weighed. Quantification of polysaccharides was performed using an established colorimetric (phenol-sulfuric acid method) assay. Three independent biofilm experiments were performed for each of the assays in duplicate.

Confocal microscopy analysis: The biofilms formed in each condition were examined using confocal laser scanning microscopy (CLSM) combined with quantitative computational analysis. Briefly, S. mutans cells were stained with 2.5 μM SYTO 9 green-fluorescent nucleic acid stain (485/498 nm; Molecular Probes Inc., Eugene, Oreg., USA), and C. albicans cells were stained with Concanavalin A (ConA) lectin conjugated with tetramethylrhodamine at 40 μg/ml (555/580 nm; Molecular Probes, Inc.), while EPS glucans were labeled with 1 μM Alexa Fluor 647-dextran conjugate (647/668 nm; Molecular Probes Inc.). The confocal images of biofilms were obtained using an upright single-photon confocal microscope (LSM800, Zeiss, Jena, Germany) with a 20× (numerical aperture, 1.0) water objective. Each component was illuminated sequentially to minimize cross-talk as follows: SYTO 9 (S. mutans) was excited using 488 nm and was collected by a 480/40 nm emission filter; ConA (C. albicans) was excited using 560 nm and was collected by a 560/40 nm emission filter; Alexa Fluor 647 (EPS) was excited using 640 nm and collected by a 670/40 nm emission filter. Biofilm images were taken at 18 h after seeding microorganisms on the sHA discs in filtered saliva supplemented with 1% (w/v) sucrose. Images were subject to the quantification of biofilm biomass and visualization. Briefly, image stacks for each channel obtained using a Zeiss LSM800 were converted to 8-bit ome.tiff files, and the COMSTAT plugin of ImageJ was used to generate values for biovolume (μm³/μm²). Biovolumes of S. mutans, C. albicans, and EPS glucans were quantified using COMSTAT2. Three independent biofilm experiments were performed for each of the assays in duplicate.

Analysis of the mechanical stability of biofilms: The mechanical stabilities of S. mutans-C. albicans biofilms with or without MDE treatment were compared using a custom-built device. Biofilms formed on sHA were placed in the disk holder of the device (FIG. 6A) and then exposed to a constant shear stress of 0.18 N/m² for 10 min. The duration of 10 min of shearing was determined to have reached a steady-state of biofilm removal. The amount of remaining biofilm dry-weight (biomass) before and after application of shear stress was determined. Also, biofilms after application of shear stress were visualized using confocal microscopy, as detailed in the previous section.

Analysis of enamel surface demineralization: Human tooth enamel blocks (4 mm×4 mm) were prepared and coated with sterile clarified whole saliva (sTE). ˜2×10⁴ CFU/mL of S. mutans and ˜2×10⁴ CFU/mL of C. albicans were grown on sTE in saliva supplemented with 1% sucrose (w/v). Briefly, biofilms were formed on enamel blocks mounted vertically at 37° C. in 5% CO₂ for 114 h. Biofilms were treated with PBS or β-mannanase as described in the ‘In vitro biofilm model’ section. Saliva medium containing 1% sucrose was replaced twice daily until the end of the experiments. Then, biofilms were gently removed, and the enamel slabs were collected for topography and surface roughness measurement. The surface topography and roughness of the enamel surface were analyzed by a nondestructive confocal contrasting method using Zeiss LSM 800 with a C Epiplan-Apochromat 50× (numerical aperture, 0.95) non-immersion objective. The images were processed using ConfoMap (Zeiss) to create 3D topography rendering and measure the surface properties in 3D. To quantify the surface demineralization, arithmetical mean height (Sₐ) was measured using ISO 25178. At least 3 independent experiments were performed for the assay.

Atomic force microscopy and analysis: Glass slides were coated with poly-L-lysine solution (0.1%; Sigma-Aldrich, St. Louis, Mo., USA) by overnight incubation. C. albicans cells were immobilized on poly-L-lysine-coated glass slides for 1 h at room temperature. Loosely adhered cells were removed by gentle washing with water, and the slide was kept hydrated prior to AFM analysis. GtfB was prepared and purified via hydroxyapatite column chromatography. AFM tips (TR400PSA, Olympus, Tokyo, Japan) were functionalized with 25 μg/mL of GtfB for 1 h at room temperature. Slides with immobilized C. albicans were incubated with optimal units of MDEs in IVIES buffer for 5 minutes at room temperature. Force measurements were then conducted under phosphate-buffered saline (HyClone Laboratories Inc., Logan, Utah, USA) using an MFP-3D AFM (Asylum Research, Santa Barbara, Calif., USA). 10×10 adhesion force maps were obtained for 12 distinct cells from 3 distinct culture preparations. Force-distance curves were analyzed using AtomicJ.

Microbicidal activity of MDEs on S. mutans and C. albicans: To assess the effect of MDEs on the growth kinetics of the microbes, overnight cultures of S. mutans and C. albicans were subcultured until each reached optical densities (600 nm) of 1.0 and 0.8, respectively. 1 mL of the subcultures were spun down and treated with MDEs for 5 min at 37° C. and pH 6.5. To remove MDEs, samples were subsequently spun down, and the supernatants were discarded. Samples were then resuspended in 1 mL of fresh LMW media and used to inoculate tubes with 9 mL of LMW media. Growth curves were monitored for 6 h by measuring OD₆₀₀ values every hour. To assess the effect of MDEs on CFU/mL of the microbes, a similar process was followed for MDE treatment. Samples were resuspended in 0.89% NaCl solution, and viable cells (CFU/mL) were counted after 48 h.

Cytotoxicity towards HGKs: HGK cells were seeded in 100 μL of KBM-2 media (Lonza Group AG, Basel, Switzerland) with 0.15 μM CaCl₂ (5000 cells/well; 96-well plate format). The next day, the media was discarded, and optimal units or 5-fold of the optimal units of MDEs were added in serum-free KBM-2 for the treatment time (1 h and 24 h). After treatment, well volumes were replaced with fresh serum-free KBM-2 media and left for a total of 24 h. The next day, 10 of 3-(4,5-Dimethyl-2-thiazolyl)-2,5-diphenyl-2H-tetrazolium bromide (MTT) reagent (Sigma-Aldrich, St. Louis, Mo., USA) was added to 90 μL of fresh serum-free KBM-2 media. Samples were left for 5 hours. Well volumes were then replaced with Dimethyl sulfoxide (DMSO, Sigma-Aldrich, St. Louis, Mo., USA). Absorbance values were read using a BioTek Elx800 (BioTek Instruments, Inc., Winooski, Vt., USA). Percentage cell viability was calculated from the absorbance readings. Three independent experiments were conducted in triplicate.

Effect of cleaved mannoproteins from C. albicans by β-mannanase on bacterial growth and pH changes: A subculture of C. albicans was centrifuged (5,500 g, 4° C., 10 min) followed by a wash with 1×PBS to remove all the nutrient media and resuspended in 3 mL of MES buffer (prewarmed at 37° C.). The suspension was split into 0.5 mL aliquots, and respective β-mannanase were added at optimal units for 5 minutes (incubate at 37° C.). C. albicans were then spun down, and supernatants were collected. ˜10⁶ of S. mutans or S. gordonii were incubated in saliva or saliva supplemented with 1% glucose or saliva supplemented with cleaved mannoproteins from C. albicans. Optical densities and pH of bacterial cultures were recorded every 2 hours.

In vivo rodent model of severe childhood caries: The therapeutic efficacy of topical MDEs treatment was assessed on a rodent caries model. 15 days-old female Sprague-Dawley rat pups were purchased with their dams from Harlan Laboratories (Madison). Upon arrival, animals were screened for S. mutans and C. albicans, and were determined not to be infected with either organism by plating oral swabs on selective media: ChromAgar (VWR International LLC, Radnor, Pa.) for C. albicans and Mitis Salivarius Agar plus Bacitracin (MSB) for S. mutans. The animals were then infected by mouth with the actively growing culture of S. mutans UA159 and C. albicans, and their infections were confirmed at 21 days via oral swabbing.

To simulate the clinical situation, a therapy consisting of 30 sec topical treatment of MDEs (or buffer) was developed. All the pups were randomly placed in equal numbers into treatment groups, and their teeth were treated topically twice daily using an applicator. The treatment groups were: (1) control (0.1M NaOAc buffer, pH 4.5) 1901, (2) 0.2% fluconazole 1902, (3) 0.2% fluconazole+10 unit of β-mannanase 1903, (4) 10 unit of β-mannanase 1904, (5) 0.2 unit of β-mannosidase 1905, and (6) 10 unit of β-mannanase+0.2 unit of β-mannosidase 1906. The treatments were blinded by placing the test agents in color-coded vials.

Each group was provided the National Institutes of Health cariogenic diet 2000 and 5% sucrose water ad libitum. This proceeded for 3 weeks (21 days). All animals were weighed weekly, and their physical appearances were noted daily. At the end of the period, the animals were sacrificed, and the jaws were surgically removed and aseptically dissected, followed by sonication to recover total oral microbiota. All jaws were defleshed, and the teeth were prepared for caries scoring according to Larson's modification of Keyes' system. Determination of the caries score of the jaws was performed by a calibrated examiner who was blind for the study by using codified samples. Furthermore, both gingival tissues were collected and processed for H&E staining for histopathological analysis by an oral pathologist at Penn Oral Pathology.

Statistical analysis: Statistical analyses were carried out using GraphPad Prism 8 using one-way ANOVA (post-hoc: Dunnett's method) and Student's t-tests where appropriate.

All patents, patent applications, publications, product descriptions, and protocols, cited in this specification are hereby incorporated by reference in their entireties. In case of a conflict in terminology, the present disclosure controls.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

What is claimed is:

1. A composition comprising:
an effective amount of a mannan degrading enzyme, wherein the effective amount is present to treat dental caries of a subject.

2. The composition of claim 1, wherein the mannan degrading enzyme is selected from the group consisting of α-mannosidase, β-mannosidase, β-mannanase, and a combination thereof.

3. The composition of claim 1, wherein the effective amount of the mannan degrading enzyme is from about 0.05 U to about 20 U.

4. The composition of claim 1, wherein the subject is younger than 6-year old.

5. The composition of claim 1, wherein the composition is formulated in a form, wherein the form is selected from the group consisting of toothpaste, a gel, a solution, a wipe, and combinations thereof.

6. The composition of claim 1, wherein the composition is configured to disrupt a formation and a development of a biofilm involved in the dental caries without damaging oral soft tissues.

7. The method for treating dental caries of a subject comprising:
administering an effective amount of a mannan degrading enzyme to a mouth of the subject, wherein the effective amount is present to treat dental caries of the subject.

8. The method of claim 7, wherein the mannan degrading enzyme is selected from the group consisting of α-mannosidase, β-mannosidase, β-mannanase, and a combination thereof.

9. The method composition of claim 7, wherein the effective amount of the mannan degrading enzyme is from about 0.05 U to about 20 U.

10. The method of claim 7, wherein the subject is younger than 6-year old.

11. The method of claim 7, wherein the mannan degrading enzyme is formulated in a form, wherein the form is selected from the group consisting of toothpaste, a gel, a solution, a wipe, and combinations thereof.

12. The method of claim 7, further comprises contacting the effective amount of a mannan degrading enzyme with a target tooth of the subject for about 5 minutes.

13. The method of claim 7, wherein a pH of the mouse is about 6 after administering the mannan degrading enzyme.

14. The method of claim 7, wherein a formation and a development of a biofilm involved in the dental caries are disrupted without damaging oral soft tissues.

15. The method of claim 7, wherein the mannan degrading enzyme is administered at least twice daily.

16. The method of claim 7, wherein the mannan is daily administered to the mouth of the subject for about three weeks.

17. The method of claim 7, wherein the administering the effective amount of the mannan degrading enzyme treats the dental caries of the subject without proliferative changes, inflammatory responses, and/or necrosis.
